(19) United States
(12) Patent Application Publication
(10) Pub. No.: US 2018/0250439 A1
(43) Pub. Date: Sep. 6, 2018

(54) HONEY ELUTING CRYOGEL FOR TISSUE ENGINEERING

(71) Applicant: Saint Louis University, St. Louis, MO (US)

(72) Inventors: Scott A. Sell, St. Louis, MO (US); Katherine R. Hixon, Davenport, IA (US); Era Jain, St. Louis, MO (US); Parin Kadakia, Vernon Hills, IL (US)

(73) Assignee: Saint Louis University, St. Louis, MO (US)

(21) Appl. No.: 15/756,864

(22) PCT Filed: Aug. 31, 2016
(86) PCT No.: PCT/US16/49655

§ 371 Date: Mar. 1, 2018

Related U.S. Application Data
(60) Provisional application No. 62/213,719, filed on Sep. 3, 2015.

(51) Int. Cl. A61L 27/36; A61L 27/56; A61L 27/52; A61L 27/58

(52) U.S. Cl. CPC A61L 27/3604; A61L 27/56; A61L 2430/02; A61L 27/58; A61L 27/52

(57) ABSTRACT

Tissue engineering structures with biologically favorable structural and chemical properties are disclosed. More particularly, the present disclosure is directed to tissue engineered structures having a cryogel scaffold and honey. The tissue engineered structures having a cryogel scaffold and honey can further include at least one biomolecule. The tissue engineered structures can be used to promote cellular chemotaxis, enhance cell proliferation, enhance extracellular matrix production, increase angiogenesis, and provide antimicrobial activity. The nature of the tissue engineered structures provides a template for cellular infiltration and guide tissue regeneration. The tissue engineered structures can be used in the treatment of dermal wounds (burns, chronic wounds, etc.) or as a tissue engineering scaffold in a wide range of applications.

### CROSS-REFERENCE TO RELATED APPLICATIONS

This application claims priority to U.S. Provisional Application No. 62/213,719, filed on Sep. 3, 2015, the disclosure of which is incorporated by reference in its entirety.

### BACKGROUND OF THE DISCLOSURE

The present disclosure relates generally to tissue engineering structures with biologically favorable structural and chemical properties. More particularly, the present disclosure is directed to cryogel scaffolds capable of incorporating and eluting honey for tissue engineering applications, and in particular bone graft substitutes. The cryogel scaffolds can be used to promote cellular chemotaxis, enhance cell proliferation, enhance extracellular matrix production, increase angiogenesis, and provide antimicrobial activity. The nature of the cryogel scaffolds provides a template for cellular infiltration and guide tissue regeneration. The cryogel scaffolds can be used in the treatment of dermal wounds (burns, chronic wounds, etc.), bone engineering and oral and maxillofacial repair.

Honey has been used medicinally for centuries, due to its inherent wound healing capacity. However, the introduction of penicillin significantly reduced the use of honey in medicinal applications. Recently, with the emergence of antibiotic-resistant bacteria and a better scientific understanding of how honey influences healing, honey (specifically active Leptospermum honey from New Zealand, known as Manuka) has once again become an acceptable product in the treatment of wounds.

The major benefit of Manuka honey lies in its potent antibacterial properties. Honey has a high osmolarity and a high sugar content, the combination of which has been shown to inhibit microbial growth. Manuka honey is also known to have a relatively low pH (3.5-4.5), which, in addition to inhibiting microbial growth, will stimulate the bactericidal actions of macrophages, and in chronic wounds reduces protease activity, increases fibroblast activity, and increases oxygenation. Hydrogen peroxide is slowly released from honey placed on a wound through the interaction of wound exudates with the honey's inherent glucose oxidase. This hydrogen peroxide is in sufficient concentration to be antibacterial, yet dilute enough to be non-toxic while promoting fibroblast proliferation and angiogenesis. Manuka honey also possesses non-peroxide antibacterial activity in what is called the Unique Manuka Factor (UMF) due to the presence of methylglyoxal.

Honey has been shown to contain a number of phenolic compounds, which are known to scavenge and remove reactive oxygen species (ROS) released by neutrophils. Honey suppressed oedema and leukocyte infiltration in a mouse model of neutrophilic inflammation. Monocytes cultured in the presence of honey produced a number of pro- and anti-inflammatory cytokines (e.g., tumor necrosis factor alpha (TNF-α), interleukin-1 beta (IL-1β), and interleukin-6 (IL-6)), which may indicate modulation towards resolution in non-healing wounds.

Bone as a whole is completely dynamic, where osteoblasts create new bone tissue and osteoclasts break down old tissue. Under natural conditions, bone regeneration following a typical fracture begins healing through the formation of a hematoma. Angiogenesis occurs and mesenchymal stem cells infiltrate the area, leading to the differentiation of chondrocytes, osteoblasts, and osteoclasts to dynamically heal the injured bone. Initially, a soft tissue callus forms for structural support until the osteoblasts start producing new bone in its place. There are cases in which this natural fracture healing is not sufficient for regenerating the injured bone. Particularly, cases including traumatic fracture, osteosarcoma, congenital malformation, vehicular accident, or military blast wounds can create problematic bone defects. Injuries such as these produce what is known as a critical size defect; that is, a defect so large that it is incapable of naturally healing during the patient's lifetime. Clinically, any bone injury in which the defect site is twice the size of the injured bone's diameter falls into that category. If left to spontaneously heal, the injury site fills with soft tissue callus without the replacement by new bone, leading to non-union.

The current treatment method for a critical size defect involves the use of a bone graft. Existing options for bone grafts include autografts, allografts, xenografts, and synthetic grafts. While autologous bone grafts are currently the favored choice due to their osteoconductive, osteoinductive, and osteogenic properties and bone regeneration, there is a major complication rate of 8.6% involved in this procedure and the patient experiences major discomfort. Further, allografts come with high costs, possible infection, and lack of donor availability. While xenografts, which are not as commonly used, offer a cheap alternative, the results are not as successful.

Accordingly, there is a major need for a bone graft substitute that can treat these critical size defects while still remaining at a low cost for the patient. To create an ideal bone graft substitute for regenerating bone, the scaffold should possess osteoconductive, osteoinductive, and osteogenic properties. Hydrogels are conventionally a very common scaffold, but the mechanical integrity and nanoporous structure of hydrogels are not advantageous for this application. It would be advantageous if the alternative bone graft substitute structure allowed for a macroporous structure for supporting cellular infiltration and tissue regeneration. It would be further advantageous if the structure was biodegradable in nature such to allow controlled degradation and integration with host tissue.

### BRIEF DESCRIPTION

In one aspect, the present disclosure is directed to a tissue engineered structure including a cryogel scaffold and honey.

In another aspect, the present disclosure is directed to a method of preparing the tissue engineered structure, the method including preparing a solution comprising honey and cryogel scaffold material; freezing the solution; and thawing the frozen solution.

In another aspect, the present disclosure is directed to a tissue engineered structure including a mineralized cryogel scaffold.

In another aspect, the present disclosure is directed to a method for promoting bone regeneration, the method comprising: implanting a tissue engineered structure comprising a mineralized cryogel scaffold.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIGS. 1A-1F show SEM images taken at 500× of: (FIG. 1A) a chitosan/gelatin (CG) hydrogel and (FIG. 1B) a CG cryogel; (FIG. 1C) a N-vinyl-2-pyrrolidone (NVP) hydrogel and (FIG. 1D) a NVP cryogel; and (FIG. 1E) a silk fibroin (SF) hydrogel and (FIG. 1F) a SF cryogel.

FIGS. 2A & 2B depict ImageJ measurements of the pore diameter (μm) (FIG. 2A) and pore area (μm²) (FIG. 2B) for chitosan/gelatin (CG), N-vinyl-2-pyrrolidone (NVP), and silk fibroin (SF) cryogels.

FIGS. 3A-3F depict μCT 3D reconstruction images. Specifically, FIG. 3A depicts a μCT 3D reconstruction image of chitosan/gelatin (CG) cryogel scaffolds. FIG. 3B depicts a μCT 3D reconstruction image of silk fibroin (SF) cryogel scaffolds. A sagittal cross section of CG cryogels (FIG. 3C) and SF cryogels (FIG. 3D) displays the inner pores for CG and SF. A sagittal cross section of CG cryogels (FIG. 3E) and SF cryogels (FIG. 3F) displays the inner pores, and the shading bar denotes the size of the pores within the scaffolds of from 0.000 mm to 0.250 mm Scale bar, 1 μm.

FIGS. 4A-4D depict μCT scans of chitosan/gelatin (CG) and silk fibroin (SF) cryogels. The scans took three readings of each type of cryogel at a threshold of 80. FIG. 4A demonstrates the amount of the total volume of the cryogel that is filled with scaffold, and FIG. 4B provides the overall connection density of the spaces. FIG. 4C depicts the average pore diameters (μm) of CG and SF cryogels, and FIG. 4D depicts the heterogeneity of the pores.

FIGS. 5A-5D depict mercury porosimetry of all dehydrated scaffold types. FIG. 5A depicts the average pore diameter (μm), FIG. 5B depicts the total pore volume (mm³/g), FIG. 5C depicts the total pore surface area (m²/g), and FIG. 5D depicts the average pore size (μm) of all three types of cryogels.

FIGS. 6A-6D depict mercury porosimetry of all hydrated scaffold types. FIG. 6A depicts the average pore diameter (μm), FIG. 6B depicts the total pore volume (mm³/g), FIG. 6C depicts the total pore surface area (m²/g), and FIG. 6D depicts the average pore size (μm) of all three types of cryogels.

FIGS. 7A-7D depict the swelling of dehydrated cryogels and hydrogels. FIG. 7A depicts the chitosan/gelatin (CG) cryogels average swelling ratio (%) vs. hydrogels. FIG. 7B depicts the N-vinyl-2-pyrrolidone (NVP) cryogels average swelling ratio (%) vs. hydrogels. FIG. 7C depicts the silk fibroin (SF) cryogels average swelling ratio (%) vs. hydrogels. FIG. 7D depicts the swelling ratio (%) of all three types of cryogels.

FIGS. 8A-8D depict ultimate compression of both cryogels and hydrogels for every material type as analyzed in Example 1. FIG. 8A depicts the average peak stress (kPa) at 50% compression. FIG. 8B depicts the average modulus (kPa) at 50% compression. FIG. 8C depicts the average peak stress (kPa) at 80% compression. FIG. 8D depicts the average modulus (kPa) at 80% compression.

FIGS. 9A-9C depict the percent stress-relaxation over 28 days of cryogels vs. hydrogels of chitosan/gelatin (CG) cryogels (FIG. 9A), N-vinyl-2-pyrrolidone (NVP) cryogels (FIG. 9B), and silk fibroin (SF) cryogels (FIG. 9C).

FIGS. 10A-10C depict the hysteresis over 28 days of cryogels vs. hydrogels for chitosan/gelatin (CG) cryogels (FIG. 10A), N-vinyl-2-pyrrolidone (NVP) cryogels (FIG. 10B), and silk fibroin (SF) cryogels (FIG. 10C).

FIGS. 11A-11D depict the absorbance (mineralization) of cryogels over 21 days for chitosan/gelatin (CG) cryogels (FIG. 11A), N-vinyl-2-pyrrolidone (NVP) cryogels (FIG. 11B), and silk fibroin (SF) cryogels (FIG. 11C), and the fold-increase of all cryogels over controls (FIG. 11D).

FIG. 12A depicts the peak stress (kPa) for all types of cryogels on days 7, 14, and 21 after mineralization as analyzed in Example 1.

FIG. 12B depicts the modulus (kPa) for all types of cryogels on days 7, 14, and 21 after mineralization as analyzed in Example 1.

FIGS. 13A-13L depict SEM images taken at 500× of a plain chitosan/gelatin (CG) cryogel (control) (FIG. 13A), day 7 mineralized CG cryogel (FIG. 13B), day 14 mineralized CG cryogel (FIG. 13C), day 21 mineralized CG cryogel (FIG. 13D), plain N-vinyl-2-pyrrolidone (NVP) cryogel (control) (FIG. 13E), day 7 mineralized NVP cryogel (FIG. 13F), day 14 mineralized NVP cryogel (FIG. 13G), day 21 mineralized NVP cryogel (FIG. 13H), plain silk fibroin (SF) cryogel (control) (FIG. 13I), day 7 mineralized SF cryogel (FIG. 13J), day 14 mineralized SF cryogel (FIG. 13K), and day 21 mineralized SF cryogel (FIG. 13L). Scale bar, 100 μm.

FIGS. 14A-14C depict the average swelling ratio (%) of chitosan/gelatin (CG) cryogels with and without honey (FIG. 14A), N-vinyl-2-pyrrolidone (NVP) cryogels with and without honey (FIG. 14B), and silk fibroin (SF) cryogels with and without honey (FIG. 14C) as analyzed in Example 2.

FIGS. 15A and 15B depict ultimate compression at 50% for chitosan/gelatin (CG) cryogels, N-vinyl-2-pyrrolidone (NVP) cryogels, and silk fibroin (SF) cryogels with and without honey. Specifically, FIG. 15A depicts peak stress (kPa) and FIG. 15B depicts modulus (kPa) of the cryogels.

FIG. 16A depicts percent stress-relaxation for silk fibroin (SF) cryogels and SF cryogels including honey as analyzed in Example 2.

FIG. 16B depicts hysteresis for silk fibroin (SF) cryogels and SF cryogels including honey as analyzed in Example 2.

FIGS. 17A-17G show SEM images taken at 500× of: (FIG. 17A) a chitosan/gelatin (CG) cryogel; (FIG. 17B) a CG cryogel with honey; (FIG. 17C) a N-vinyl-2-pyrrolidone (NVP) cryogel; (FIG. 17D) a NVP cryogel with honey; (FIG. 17E) a silk fibroin (SF) cryogel; (FIG. 17F) a SF cryogel with 1% honey; and (FIG. 17G) a SF cryogel with 5% honey. Scale bar, 100 μm.

FIG. 18A depicts SEM image measurements of pore diameter for silk cryogels and silk cryogels with 1% and 5% honey as analyzed in Example 2.

FIG. 18B depicts SEM image measurements of pore area for silk cryogels and silk cryogels with 1% and 5% honey as analyzed in Example 2.

FIG. 19 depicts mineralization with simulated body fluid (SBF) of chitosan gelatin cryogels, NVP cryogels and silk cryogels incorporated with honey for days 7, 14, and 21.

FIGS. 20A-20H depict SEM images of gelatin cryogels (FIG. 20A), SF cryogels (FIG. 20B), gelatin cryogel with 1% honey (FIG. 20C), SF cryogel with 1% honey (FIG. 20D), gelatin cryogel with 5% honey (FIG. 20E), SF cryogel with 5% honey (FIG. 20F), gelatin cryogel with 10% honey (FIG. 20G) and SF cryogel with 10% honey (FIG. 20H). Scale bar, 100 μm.

FIGS. 21A-21H depict μCT 3D reconstruction images of the cryogels where a sagittal cross section displays the inner pores for gelatin cryogel (FIG. 21A), SF cryogel (FIG. 21B), gelatin cryogel with 1% honey (FIG. 21C), SF cryogel with 1% honey (FIG. 21D), gelatin cryogel with 5% honey (FIG. 21E), SF cryogel with 5% honey (FIG. 21F), gelatin cryogel with 10% honey (FIG. 21G), and SF cryogel with 10% honey (FIG. 21H). Note that the shading bar denotes the pore size within each scaffold. Scale bar, 1.0 mm.

FIGS. 22A-22D depict μCT scans of gelatin cryogels and silk fibroin (SF) cryogels with 0%-10% honey (n=3) taken at a threshold of 80. The scans provide the void diameter (μm) (FIG. 22A), diameter heterogeneity (FIG. 22B), pore connection density (1/mm³) (FIG. 22C), and void/total volume ratio (FIG. 22D).

FIGS. 23A and 23B depict average swelling ratio (%) for gelatin cryogels with 0%-10% honey (FIG. 23A) and SF cryogels with 0%-10% honey (FIG. 23B).

FIGS. 24A and 24B depict average peak stress (kPa) for gelatin cryogels with 0%-10% honey and SF cryogels with 0%-10% honey (FIG. 24A) and average modulus (kPa) for gelatin cryogels with 0%-10% honey and SF cryogels with 0%-10% honey (FIG. 24B).

FIGS. 25A-25F depict bacterial clearance of Group B Streptococcus (GBS) in gelatin cryogels (FIG. 25A) and SF cryogels (FIG. 25B), Escherichia coli (E. coli) in gelatin cryogels (FIG. 25C) and SF cryogels (FIG. 25D), and Staphylococcus (Staph) for gelatin cryogels (FIG. 25E) and SF cryogels (FIG. 25F) with 0%-10% honey. “Disc” refers to a sterile disc with no honey; “HC” refers to Manuka honey controls that were placed directly onto the bacteria.

FIGS. 26A and 26B depict cellular proliferation of MG-63 cells on gelatin cryogels and SF cryogels with 0%-10% honey and sterilized in 70% ethanol (FIG. 26A) or peracetic acid (FIG. 26B). Note that TCP and honey media were used as controls.

FIGS. 27A-27H depict SEM images of mineralized cryogels obtained through dunking. Gelatin cryogels (FIG. 27A), SF cryogels (FIG. 27B), Gelatin cryogels with 1% honey (FIG. 27C), SF cryogels with 1% honey (FIG. 27D), Gelatin cryogels with 5% honey (FIG. 27E), SF cryogels with 5% honey (FIG. 27F), Gelatin cryogels with 10% honey (FIG. 27G), and SF cryogels with 10% honey (FIG. 27H). All images were taken at 500×. Scale bar, 100 μm.

FIG. 28 depicts gelatin cryogel mineralization through dunking for 0 hour-10 hours.

FIGS. 29A and 29B depict glucose release over 14 days from mineralized gelatin cryogels (FIG. 29A) and mineralized SF cryogels (FIG. 29B) with honey and sterilized with ethanol or peracetic acid (PAA).

FIGS. 30A-30D depict fold increase of bacterial clearance of Escherichia coli (E. coli) in gelatin cryogels (FIG. 30A) and SF cryogels (FIG. 30B) and Staphylococcus (Staph) broth in gelatin cryogels (FIG. 30C) and SF cryogels (FIG. 30D) as effected by the presence of 1%-10% honey.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred materials and methods are described below.

In accordance with the present disclosure, tissue engineered structures having a cryogel scaffold incorporating honey therein and methods for preparing the tissue engineered structures are described. The tissue engineered structures can further include at least one biomolecule. The tissue engineered structures can be used to promote cellular chemotaxis, enhance cell proliferation, enhance extracellular matrix production, increase angiogenesis, and provide antimicrobial activity. The nature of the tissue engineered structures provides a template for cellular infiltration and can guide tissue regeneration. The tissue engineered structures can be used in the treatment of dermal wounds (burns, chronic wounds, etc.) or as a tissue engineering structure in a wide range of applications such as for bone engineering and oral and maxillofacial repair.

Cryogelation is a technique in which the controlled freezing and thawing of the polymer creates a spongy, macroporous structure with ideal mechanical properties. The polymer or monomer solution is frozen in a controlled manner such that there is ice crystal formation throughout the gel prior to polymerization. When slowly thawed at a controlled temperature, these ice crystals melt leaving a macroporous structure ideal for cellular infiltration. Additionally, this particular method of formation leaves the resulting polymer structure with increased mechanical stability and a sponge-like consistency. Structurally, these cryogel scaffolds offer an ideal pore size, distribution, and interconnectivity for tissue engineering.

### Tissue Engineered Structures

In one aspect, the present disclosure is directed to tissue engineered structures. The tissue engineered structures include a cryogel scaffold and honey.

Materials used to prepare the cryogel scaffold can be any polymeric materials known in the art. Suitable cryogel scaffolds can be biodegradable (also referred to herein as “bioresorbable”) material, non-biodegradable material and combinations thereof. As used herein, “bioresorbable” and “biodegradable” are used interchangeably herein to refer to a material that is biocompatible as well as degradable and/or absorbable by a subject. Biodegradable material is intended to be broken down (usually gradually) by the body of an animal, e.g. a mammal. A bioresorbable material is intended to be absorbed or resorbed by the body of a subject, such that it eventually becomes essentially non-detectable at the site of application.

Materials for the cryogel scaffold can be a synthetic polymer, a natural protein and combinations thereof. Suitable synthetic polymers can be, for example, polycaprolactone (PCL), polydioxanone (PDO), poly (glycolic acid) (PGA), poly(L-lactic acid) (PLA), poly(lactide-co-glycolide) (PLGA), poly(L-lactide) (PLLA), poly(D,L-lactide) (P(DLLA)), poly(ethylene glycol) (PEG), poly(ε-caprolactone) (PCL), montmorillonite (MMT), poly(L-lactide-co-ε-caprolactone) (P(LLA-CL)), poly(ε-caprolactone-co-ethyl ethylene phosphate) (P(CL-EEP)), poly[bis(p-methylphenoxy) phosphazene] (PNmPh), poly(3-hydroxybutyrate-co-3-hydroxyvalerate) (PHBV), poly(ester urethane) urea (PEUU), poly(p-dioxanone) (PPDO), polyurethane (PU), polyethylene terephthalate (PET), poly(ethylene-co-vinylacetate) (PEVA), poly(ethylene oxide) (PEO), poly(phosphazene), poly(ethylene-co-vinyl alcohol), polymer nanoclay nanocomposites, a halogenated polymer solution containing metal compounds (e.g., graphite), poly(ethylenimine), grafted cellulosics, poly(ethyleneoxide), poly vinylpyrrolidone, polystyrene (PS) and combinations thereof.

Suitable natural proteins can be, for example, silk fibroin, collagen, elastin, hyaluronic acid, gelatin, fibrinogen, chitin, chitosan, fibronectin and combinations thereof.

As noted above, the cryogel scaffold is porous. Suitable pore sizes can range from about 15 μm to about 500 μm, including from about 75 μm to about 150 μm, and including from about 100 μm to about 125 μm. Pore size can be determined, for example, by scanning electron microscopy. Porosity and pore size allow cells to migrate into and through pores and infiltrate into the scaffold, allow culture medium circulation into and through pores, and allow exchange of nutrients and metabolic waste.

Any suitable honey can be incorporated into the cryogel scaffold. Particularly suitable honey can be, for example, Manuka honey, medical-grade Medihoney, which is a Manuka honey product, and combinations thereof. Manuka honey is a specifically active Leptospermum honey from New Zealand. Suitable honeys can further include those having a high osmolarity and a high sugar content. Suitable honeys can further include those having an acidic pH. Particularly suitable acidic pH honeys are those having a low pH such as, for example, a pH of about 3.5 to about 4.5.

In another aspect, the tissue engineered structures include a cryogel scaffold and honey, as described herein, and further include at least one biomolecule.

Suitable biomolecules can be, for example, growth factors, cytokines, bioactive lipids, immunoglobulins, and combinations thereof. Particularly suitable biomolecules can be, for example, platelet derived growth factor (PDGF), transforming growth factor beta (TGFβ), vascular endothelial growth factor (VEGF), fibroblast growth factor (FGF), epidermal growth factor (EGF), human growth factor (HGF), bone morphogenetic proteins (BMPs; e.g., BMP1, BMP2, BMP3, BMP4, BMP5, BMP6, BMP7, and BMP8a), insulin-like growth factors (e.g., IGF-1 and IGF-2), keratinocyte growth factor, connective tissue growth factor, chemotactic proteins, sphingosine 1-phosphate (S1P), various macrophage and monocyte mediators such as RANTES (Regulated upon Activation, Normal T-cell Expressed, and Secreted), tumor necrosis factor α (TNF α), interferon gamma (IFNγ), and granulocyte-macrophage colony stimulating factor (GM-CSF), lipoxin and combinations thereof. Suitable cytokines can be, for example, interleukins (e.g., IL-1-IL-36) and interferons (e.g., interferon type I, interferon type II, interferon type III).

The biomolecule can also be a preparation rich in growth factors (PRGF). PRGF can be prepared from blood or platelet rich plasma (PRP). To prepare PRGF, blood can be used to create PRP using methods known to those skilled in the art. For example, the HARVEST® SMARTPREP® 2 kit (Harvest Technologies Corp., Plymouth, Mass.) is a commercially available centrifugation system to create PRP. After obtaining PRP, the PRP is then subjected to a freeze-thaw-freeze (FTF) cycle for cell lysis. The FTF cycle can be performed by placing PRP in a −70° C. freezer, followed by thawing in a 37° C. water bath, and then returned to the −70° C. freezer. The frozen PRP is then lyophilized to create a dry PRGF powder that can be finely ground in a mortar and pestle prior to use.

In another aspect, the cryogel scaffolds can include a plurality of cells. Cell types that can be used are, for example, endothelial cells, macrophages, adipose-derived stem cells, mesenchymal stem cells, embryonic stem cells, ligament fibroblasts, tendon fibroblasts, muscle fibroblasts, dermal fibroblasts, muscle cells and combinations thereof. The cells can be autologous cells, allogeneic cells, or xenogeneic cells. “Autologous cells” refers to cells that are donated and received by the same subject. For example, cells are obtained from subject A, incorporated into the scaffold support, and the cell-laden scaffold support can be implanted into subject A. “Allogeneic cells” refers to cells that are donated by a subject that is different from the recipient subject; however, the donor subject and recipient subject are from the same species. For example, cells are obtained from subject A, incorporated into the scaffold support, and the cell-laden scaffold support is implanted into subject B. “Xenogeneic cells” refers to cells that are obtained from or donated by a species that is different than the recipient. For example, cells are obtained from species A, incorporated into the scaffold support, and the cell-laden scaffold support is implanted into species B.

In another aspect, the cryogel scaffolds can further be coated with a cell adhesion molecule. The cell adhesion molecule coating the cryogel scaffolds contacts polymers making up the scaffold. The cell adhesion molecule can be, for example, fibronectin, vitronectin, collagen, RGD (arginine-glycine-aspartic acid) peptide, LDV (leucine-aspartic acid-valine) peptide, laminin and combinations thereof. Unique physical characteristics of cryogels enhance adsorption of cell adhesion molecules, induce favorable cell to extracellular matrix interactions, promote in vivo-like three-dimensional adhesion and activate cell signaling pathways, maintain cell phenotype, and support cell differentiation.

In another aspect, the cryogel scaffolds can further be coated with other molecules such as, for example, recombinant and chemically synthesized proteins and peptides and nucleic acids (DNA and RNA).

### Mineralized Cryogel Scaffolds

In another aspect, the present disclosure is directed to a tissue engineered structure including a mineralized cryogel scaffold.

In one embodiment, the mineralized cryogel scaffold further includes honey. Any suitable honey can be incorporated into the cryogel scaffold. Particularly suitable honey can be, for example, Manuka honey, medical-grade Medihoney, which is a Manuka honey product, and combinations thereof. Manuka honey is a specifically active Leptospermum honey from New Zealand. Suitable honeys can further include those having a high osmolarity and a high sugar content. Suitable honeys can further include those having an acidic pH. Particularly suitable acidic pH honeys are those having a low pH such as, for example, a pH of about 3.5 to about 4.5. Honey can be incorporated into and/or applied to the mineralized cryogel scaffold by dunking, dipping, pouring, spraying, brushing, soaking, and other application methods. Honey can also be incorporated into the mineralized cryogel scaffold by preparing a solution having a cryogel scaffold material; adding honey to the solution; freezing the solution including the cryogel scaffold material and honey; thawing the frozen solution; re-freezing the thawed solution; and collecting the cryogel scaffold having honey incorporated therein. The cryogel scaffold having honey incorporated can then be contacted with a mineralizing solution such as, for example, simulated body fluid, as described herein.

Materials used to prepare the cryogel scaffold can be any polymeric materials known in the art as described herein. Suitable cryogel scaffolds can be biodegradable (also referred to herein as “bioresorbable”) material, non-biodegradable material and combinations thereof as described herein. Suitable materials for the cryogel scaffold can be a synthetic polymer, a natural protein and combinations thereof as described herein.

As noted above, the cryogel scaffold is porous as described herein. Suitable pore sizes can range from about 15 μm to about 500 μm, including from about 75 μm to about 150 μm, and including from about 100 μm to about 125 μm.

The mineralized cryogel scaffold can further include at least one biomolecule as described herein. The biomolecule of the mineralized cryogel scaffolds can be incorporated into the scaffold, into the mineral coating, on the mineral coating, and combinations thereof. The mineralized cryogel scaffolds can further be coated with a cell adhesion molecule as described herein. The cell adhesion molecule of the mineralized cryogel scaffolds can be incorporated into the scaffold, into the mineral coating, on the mineral coating, and combinations thereof. The mineralized cryogel scaffolds can further be coated with other molecules such as, for example, recombinant and chemically synthesized proteins and peptides and nucleic acids (DNA and RNA). The recombinant and chemically synthesized proteins and peptides and nucleic acids (DNA and RNA) of the mineralized cryogel scaffolds can be incorporated into the scaffold, into the mineral coating, on the mineral coating, and combinations thereof.

The mineralized cryogel scaffolds can further include a plurality of cells as described herein.

### Methods of Preparing Tissue Engineered Structures

In another aspect, the present disclosure is directed to a method of preparing a tissue engineered structure, wherein the tissue engineered structure has at least a cryogel scaffold and honey. The method includes preparing a solution having a cryogel scaffold material; adding honey to the solution; freezing the solution including the cryogel scaffold material and honey; thawing the frozen solution; re-freezing the thawed solution; and collecting the cryogel scaffold having honey incorporated therein.

The tissue engineered scaffold can be prepared using suitable cryogel scaffold materials and honeys as described herein.

The solution is prepared by dispersing cryogel scaffold material in a solvent. The cryogel scaffold material can be dispersed in the solvent using methods known by those skilled in the art such as, for example, sonicating, vortexing, mixing, stirring and combinations thereof. A particularly suitable method is by sonication. The amount of cryogel scaffold material to be dispersed in the solvent can be, for example, about 1% (weight/volume) to about 10% (weight/volume). One skilled in the art would readily understand that the amount of cryogel scaffold material to be added can depend on the type of scaffold material and its particular characteristics.

Any suitable solvent known by those skilled in the art may be used that is capable of dissolving the cryogel scaffold materials. Suitable solvents can be, for example, acetic acid, deionized water, N,N-dimethyl formamide (DMF), tetrohydrofuran (THF), N,N-dimethyl acetamide (DMAc), chloroform, methylene chloride, dioxane, ethanol, 1,1,1,3,3,3 hexafluoroisopropanol (HFP) and combinations thereof. A particularly suitable solvent is 1,1,1,3,3,3 hexafluoroisopropanol (HFP).

Upon dispersion of the cryogel scaffold material in the solvent, honey is added to the solvent solution. The amount of honey to be added can range from about 1% (by volume/volume) to about 50% (by volume/volume), and, for example, about 5% (by volume/volume) and about 10% (by volume/volume).

After dispersion of the cryogel scaffold material and honey into the solvent solution, the mixture undergoes a freeze/thaw cycle. For example, in particularly suitable embodiments, the mixtures undergo a freeze/thaw cycle between −20° C. and 4° C., respectively. More particularly, for example, the cycle begins with a freeze of 24 hours in a methanol bath at −20° C. followed by a thaw in a water bath for 24 hours at 4° C.

In another aspect, the present disclosure is directed to a method of preparing a tissue engineered structure, wherein the tissue engineered structure has a cryogel scaffold material; honey; and at least one biomolecule. In one embodiment, the method includes preparing a solution having the cryogel scaffold material, honey and a solvent dispersed therein; adding at least one biomolecule to the solution; and subjecting the solution to a freeze/thaw cycle. Additionally or alternatively, a honey-containing cryogel can first be prepared, followed by coating the cryogel with a biomolecule. The honey-containing cryogel can be soaked in a solution including the biomolecule. Without being bound by theory, it is believed that all surfaces of the cryogel will be exposed to the solution containing the biomolecule, including, for example, within pores and channels of the cryogel.

The tissue engineered structure can be prepared using suitable cryogel scaffold materials, honey, biomolecules and solvents as described herein.

In another aspect, the present disclosure is directed to a method of preparing a tissue engineered structure, wherein the tissue engineered structure comprises a mineralized cryogel scaffold. The method includes preparing a solution having a cryogel scaffold material; freezing the solution including the cryogel scaffold material; thawing the frozen solution; re-freezing the thawed solution; collecting the cryogel scaffold; contacting the cryogel scaffold with a mineralizing solution for a sufficient time to form a mineral on the cryogel scaffold; and collecting the mineralized cryogel scaffold.

In another aspect, the present disclosure is directed to a method of preparing a tissue engineered structure, wherein the tissue engineered structure comprises a mineralized cryogel scaffold and honey. The method includes preparing a solution having a cryogel scaffold material; adding honey to the solution; freezing the solution including the cryogel scaffold material and honey; thawing the frozen solution; re-freezing the thawed solution; collecting the cryogel scaffold having honey; contacting the cryogel scaffold having honey with a mineralizing solution for a sufficient time to form a mineral on the cryogel scaffold; and collecting the mineralized cryogel scaffold.

In another aspect, the present disclosure is directed to a method of preparing a tissue engineered structure, wherein the tissue engineered structure comprises a mineralized cryogel scaffold and honey. The method includes preparing a solution having a cryogel scaffold material; freezing the solution including the cryogel scaffold material; thawing the frozen solution; re-freezing the thawed solution; collecting the cryogel scaffold; contacting the cryogel scaffold with a mineralizing solution for a sufficient time to form a mineral on the cryogel scaffold; collecting the mineralized cryogel scaffold; and contacting the mineralized cryogel scaffold with honey.

Particularly suitable mineralizing solutions include Simulated Body Fluid (SBF) as described in Oyane, A. et al. Preparation and assessment of revised simulated body fluids. Journal of biomedical materials research. Part A 65, 188-195, doi:10.1002/jbm.a.10482 (2003).

The cryogel scaffolds can further include at least one biomolecule as described herein. The biomolecule of the cryogel scaffolds can be incorporated into the scaffold, into the mineral coating, on the mineral coating, and combinations thereof. The cryogel scaffolds can further be coated with a cell adhesion molecule as described herein. The cell adhesion molecule of the cryogel scaffolds can be incorporated into the scaffold, into the mineral coating, on the mineral coating, and combinations thereof. The cryogel scaffolds can further be coated with other molecules such as, for example, recombinant and chemically synthesized proteins and peptides and nucleic acids (DNA and RNA). The recombinant and chemically synthesized proteins and peptides and nucleic acids (DNA and RNA) of the cryogel scaffolds can be incorporated into the scaffold, into the mineral coating, on the mineral coating, and combinations thereof.

Materials used to prepare the cryogel scaffold can be any polymeric materials known in the art and described herein. Suitable cryogel scaffolds can be biodegradable (also referred to herein as “bioresorbable”) material, non-biodegradable material and combinations thereof as described herein. The solution is prepared by dispersing cryogel scaffold material in a solvent as described herein. Any suitable solvent known by those skilled in the art may be used that is capable of dissolving the cryogel scaffold materials as described herein. Suitable solvents include those described herein.

After dispersion of the cryogel scaffold material and honey into the solvent solution, the mixture undergoes a freeze/thaw cycle as described herein.

The disclosure will be more fully understood upon consideration of the following non-limiting Examples.

### EXAMPLES

Cryogel Characterization Methods

1. Scanning Electron Microscope (SEM)

All samples were air dried for 24 hours, mounted on an aluminum stub, and sputter coated (SoftComp, Bal-Tec SCD 005) in gold at 20 mA for 360 seconds. SEM (Zeiss, Evo LS15) images were obtained to examine the morphology at 100×, 200×, 500×, and 1kX under high vacuum. Following imaging, measurements of each sample type were done using ImageJ (NIH). For this technique, the scale bar was set with respect to the image pixel size. 60 measurements of pore diameter and area were randomly taken by measuring the long diameter of the pore and tracing the pore, respectively.

2. μCT

To further evaluate pore size and interconnectivity a μCT (μCT 35, Scanco Medical, Wayne, Pa.; X-ray tube potential 45 kVp, integration time 600 ms, X-ray intensity 4 W, isotropic voxel size 7 um, frame averaging 1, projections 500, medium resolution scan) was used. Three samples of each type of cryogel scaffold were scanned at thresholds of 50, 60, 70, 80, 90, 100, and 110. A threshold of 80 was chosen to record measurements based upon user experience and pore clarity. The average pore diameter (μm), scaffold connection density (1/mm³), and total ratio filled with scaffold were obtained.

3. Mercury Intrusion Porosimetry

The overall porosity of the cryogel scaffold samples were also examined using mercury intrusion porosimetry. A Quantachrome Instruments Ultrapyc 1200e pycnometer (model no. MUPY-31) was employed. Density analysis was completed according to manufacturer's protocol using ultrapure helium gas and a maximum pressure of 3 psig. For each sample, the sample weight was entered into the instrument's software and the pycnometer completed a total of 9 runs, averaging the 5 runs with the best standard deviations. Mercury intrusion porosimetry was performed to evaluate the porosity of the different sample types. Specifically, a Thermo Scientific Pascal 140 Series porosimeter with elemental mercury (ALFA AESAR® 99.9% redistilled mercury) was used for these samples. The samples underwent pressurized mercury intrusion according to manufacturer's instrument protocol with the use of Dilatometer 44 (mercury height: 90.5 mm, stem mercury height: 64.5 mm, filling volume: 456 mm³, cone height: 21.0 mm, electrode gap: 5.0 mm, stem radius: 1.5 mm). The individual sample's weight and density (previously obtained via the pycnometer) were entered prior to mercury filling. After the sample was loaded into the dilatometer, the dilatometer was filled with mercury to its filling volume and then pressurized to the instrument's maximum pressure of 400 kPa. After completion of the mercury intrusion, data regarding the sample's porosity was collected and used in further sample analysis. The process was repeated for both dry and hydrated samples. The Silk Fibroin (SF) samples were hydrated in DI water for 48 hours prior to testing. The chitosan/gelatin (CG) and N-Vinyl-2-pyrrolidone (NVP) samples were hydrated in deionizing (DI) water for 10 minutes prior to resting. For the hydrated samples, the sample type's respective densities were maintained, but their hydrated weight was used as their respective sample weight. The hydrogels porosity did not allow them to be tested using this procedure.

4. Swelling

To test shape retention and rehydration potential of the constructs, a swelling test was performed. Three types of samples of each hydrogel and cryogel were completely dehydrated for 48 hours. After being placed in DI water, each sample was removed and weighed at time points 2 minutes, 4 minutes, 10 minutes, 20 minutes, 40 minutes, 1 hour, 2 hours, 4 hours, and 24 hours. The average swelling ratio, taking into account the original dry weight of each sample, was recorded using the equation below (1):

Swelling Ratio=(Ws−Wd)/Wd (1)

where Ws is the swelled gel weight and Wd is the dry gel weight.

5. Ultimate Compression

To test the mechanical integrity of the hydrogels and cryogels, ultimate compression at both 50% and 80% was completed for each material type (n=6). To do this, a Mechanical Testing System (MTS Criterion Model 42, MTS Systems Corporation) was fitted with a 100 N load cell. A test rate of 10 mm/min, preload of 0.05 N, data acquisition rate of 10 Hz, and preload speed of 1 mm/min was used to compress each sample to either 50 or 80% of its original volume, taking into account both the diameter and thickness. Data integration was completed using MTS TW Elite software to record both the peak stress (kPa) and modulus (kPa).

6. Cyclic Loading with Degradation

Five samples of each type of hydrogel and cryogel scaffold were cyclically loaded 20 times using the MTS system mentioned above and then placed in sterile phosphate buffered saline (PBS). The samples underwent cyclic loading on days 1, 3, 7, 14, 21, and 28 and placed in fresh PBS after each test. Cyclic loading parameters included a preload speed of 2.54 mm/min, plate separation force of 4.448 N, test speed of 10 mm/min, plate separation speed of 10 mm/min, hold times of 0 seconds, preload of 0.05 N, and compression of 20% and 5%. Data integration was completed using MTS TW Elite software and the percent stress-relaxation and hysteresis were found using a premade Matlab program.

7. Cellular Infiltration

All cryogels were sterilized in 70% ethanol (Fisher Scientific, NJ) on a shaker plate for 30 minutes, followed by an additional 30 minutes in 70% ethanol in the fume hood, and three 10 minute washes with sterile PBS. Half of the scaffolds were then soaked in complete media composed of Dulbecco's Modified Eagle's Medium (DMEM) with 4.5 g/L Glucose & L-Glutamine (Lonza, Md.), 10% fetal bovine serum (FBS) (Biowest, Tex.) and 1% penicillin-streptomycin solution (Hyclone, Pa.) for an additional hour to allow for protein absorption and potentially enhanced cellular attachment. Once sterilized, all scaffolds were placed in a 48-well plate (Falcon, N.Y.). 100 μL of media containing 50,000 human bone osteosarcoma-derived cells (MG-63; ATCC, VA) were seeded onto each scaffold by slowly dripping the solution on the top. Once seeded, the 48-well plates were incubated for two hours at 37° C. and 5% CO₂ to allow the attachment of the cells. At this time, an additional 175 μL of complete media was added so that all samples were completely submerged. The media was changed every two to three days from around the scaffold. The cryogel scaffolds were removed at days 7, 14, 21, and 28 and placed in formalin (Protocol, Mich.). Half of each scaffold was embedded in paraffin and sectioned using a microtome. These sections were then stained with DAPI to observe cellular infiltration over the various time points.

8. Mineralization from MG-63 Cells

The other half of the scaffolds from days 7, 14, 21, and 28 mentioned above were stained with alizarin red. This allowed for the detection of any presence of mineralization. The protocol for alizarin red staining was followed as noted in Rodriguez, I. A. et al., A preliminary evaluation of lyophilized gelatin sponges, enhanced with platelet-rich plasma, hydroxyapatite and chitin whiskers for bone regeneration. Cells 2, 244-265, doi:10.3390/cells2020244 (2013). Sections of the scaffold were also SEM imaged to detect any surface and internal mineralization.

9. Mineralization using Simulated Body Fluid (SBF)

Seven samples of each type of cryogel scaffold were obtained as previously described. All cryogel scaffolds were sterilized in 70% ethanol on a shaker plate for 30 minutes, 70% ethanol in the sterile hood for 30 minutes, and three 10 minute washes with sterile PBS. Simulated Body Fluid (SBF) was prepared as c-SBF as described in Oyane, A. et al. Preparation and assessment of revised simulated body fluids. Journal of biomedical materials research. Part A 65, 188-195, doi:10.1002/jbm.a.10482 (2003) no less than 24 hours prior to use and kept in the incubator at 37° C. All scaffolds were placed in a 48-well plate with 1 mL of SBF for 7, 14, and 21 days. The SBF was replaced with fresh SBF every seven days. At each time point, all samples were rinsed with water and one sample was dried and SEM imaged, three samples underwent 50% ultimate compression, and three samples were stained with alizarin red to detect mineralization. The protocol for alizarin red staining was again followed from Rodriguez, I. A. et al., Cells 2, 244-265, doi:10.3390/cells2020244 (2013).

### Example 1

In this Example, cryogel scaffolds containing chitosan and gelatin (CG), cryogel scaffolds containing N-vinyl-2-pyrrolidone (NVP), and cryogel scaffolds containing silk fibroin (SF) were prepared. The structural and physical characteristics of the cryogel scaffolds were then analyzed and compared to their corresponding hydrogel scaffolds made of the same polymers.

To prepare the cryogel scaffolds containing chitosan and gelatin (CG cryogel scaffolds), a 10-mL solution (pH 2.5) of 1% acetic acid (Fisher Scientific, NJ) (9.9 mL of water and 100 μL acetic acid) was made. 80 mg of low viscosity chitosan (MP Biomedicals, OH) was ultraviolet (UV) sterilized and dissolved in 8 mL of the 1% acetic acid solution and then placed into a scintillation vial.

The scintillation vial was placed on a spinner until thoroughly mixed (˜30 minutes), and 320 mg of gelatin (from cold water fish skin) (Sigma-Aldrich, MO)) was UV sterilized and added to the above vial of solution. To avoid the formation of bubbles, the vial was placed on a mechanical shaker for approximately an hour until the gelatin was completely dissolved.

The remaining 2 mL of 1% acetic acid was combined with glutaraldehyde (Sigma-Aldrich, MO) to create a 1% glutaraldehyde solution. Specifically, a 1% solution of glutaraldehyde was prepared by diluting glutaraldehyde in 2 mL of 1% acetic acid (e.g., from a 50% glutaraldehyde, take out 100 μL from leftover 2 mL of 1% acetic acid and add 100 μL of glutaraldehyde).

Both prepared solutions were then cooled at 4° C. for 1 hour. After cooling, the solutions were mixed by slowly pouring between the vials and then pouring the solution into pre-cooled (−20° C.) 3 cc syringes (BD, N.J.). Parafilm (Bemis, Wis.) was used to seal off either side of the syringe and the filled syringes were immediately placed in a −20° C. methanol bath (Fisher Scientific, NJ). After at least 16 hours, the CG cryogel scaffolds were taken out, the parafilm removed, and the gel filled syringes placed in room temperature, sterile water until thawed.

To create a corresponding CG hydrogel, the previous procedure relative to the formation of the solutions was followed. Instead of the solutions being mixed and subjected to freeze/thaw cycles, the combined polymer solution was placed at room temperature for 16 hours to ensure complete hydrogel formation.

To prepare the cryogel scaffolds containing N-vinyl-2-pyrrolidone (NVP), 500 μL of NVP (Acros, N.J.) was added to approximately 7 mL of deionizing (DI) water in a 50-mL tube (Fisher Scientific, NJ) and mixed using a vortex. Once mixed, 0.15 g of Bis Acrylamide (Promega, Wis.) was added, and the total volume was brought up to 10 mL with additional DI water.

The solution went through freezing/thawing cycles (freeze solution (−20° C.) and thaw solution (4° C.)) for different time intervals (i.e., freeze for 30 minutes and thaw for 15 minutes, freeze for another 30 minutes, thaw for 10 minutes and freeze for an hour). The solution was then stored at 4° C. for an hour until it had thawed completely.

The thawed solution was then purged with argon to displace dissolved oxygen (˜2 minutes) and free radical polymerization was initiated by adding tetramethylethylenediamine (TEMED (20 μL)) and a premade solution of ammonium persulfate (APS (10 mg in 100 μL DI water) (Acros, NJ)). The solution was vortexed between additions of these additives and then poured into pre-cooled (−20° C.) 3 cc syringes. Parafilm was used to seal off either side of the syringe and filled syringes were immediately placed in a −20° C. methanol bath. After at least 16 hours, the cryogel scaffolds were removed, the parafilm removed, and the gel filled syringes placed in room temperature water until thawed.

To create a corresponding NVP hydrogel, the previous procedure relative to the formation of the solutions was followed. Instead of the solutions being mixed and subjected to freeze/thaw cycles, the combined polymer solution was placed at room temperature for 16 hours to ensure complete hydrogel formation.

The following process was used to prepare the cryogel scaffolds containing silk fibroin (SF). First, solutions of SF were prepared. To prepare solutions of silk fibroin (SF), silk cocoons were cut into four small pieces and dead silk worms were discarded. Five grams of sliced cocoons and 4.24 grams of Na₂CO₃ were added to 2 L of boiling distilled water for 30 minutes. This step was required to remove the sericin protein of silk. Next, boiled silk cocoons underwent three 20-minute rinses with 1 L distilled water and then were dried at room temperature. Dried silk fibers were subsequently dissolved in a 9.3 M LiBr solution at 60° C. for 4 hours. Afterwards, the dissolved fibers were dialyzed (3.5 kD MWCO, Spectra/Por) against 1 L of distilled water at 4° C. for three days, with the water being changed every hour for the first 4 hours, twice the second day (morning and evening), and once the third day (morning). To eliminate any impurities, these aqueous solutions were centrifuged twice at 8500 rpm for 20 minutes. In cases were the DI water was added for dilution purposes, solution was slowly pipetted to allow for even mixing (and to ensure limitation of the formation of bubbles). Final silk solutions were stored at 4° C. and utilized within two weeks of fabrication. Silk fibroin concentration was determined by drying out a known volume of silk and was found to be approximately 4% (w/v) following dialysis. To increase the silk concentration, solutions were dialyzed (3.5 kD MWCO, Spectra/Por) against a 10% (w/v) PEG (10,000 g/mol) solution for 3 to 4 hours.

0.5 mL of the prepared aqueous silk solution was heated at 60° C. for 24 hours. The silk was weighed and the final concentration was calculated as shown below:

Concentration of Silk (w/v %)=m_{f}−mᵢ*200 (2)

500 μL of silk solution was then added to 2-mL centrifuge tubes. Holding the tubes steady in a small ice bath, silk solutions were probe sonicated with a Fisher Sonic Dismembrator Model 100 for 30 seconds at a probe intensity of 2. Following sonication, the tubes were stored in a methanol bath at −20° C. for 24 hours. The resulting SF cryogel scaffolds were thawed in distilled water for 24 hours at room temperature before use.

Silk fibroin (SF) hydrogel scaffolds were made with a similar process except these scaffolds were stored at room temperature for 24 hours instead of −20° C. The concentration of silk fibroin solution used to make these cryogels was 4.5% (w/v).

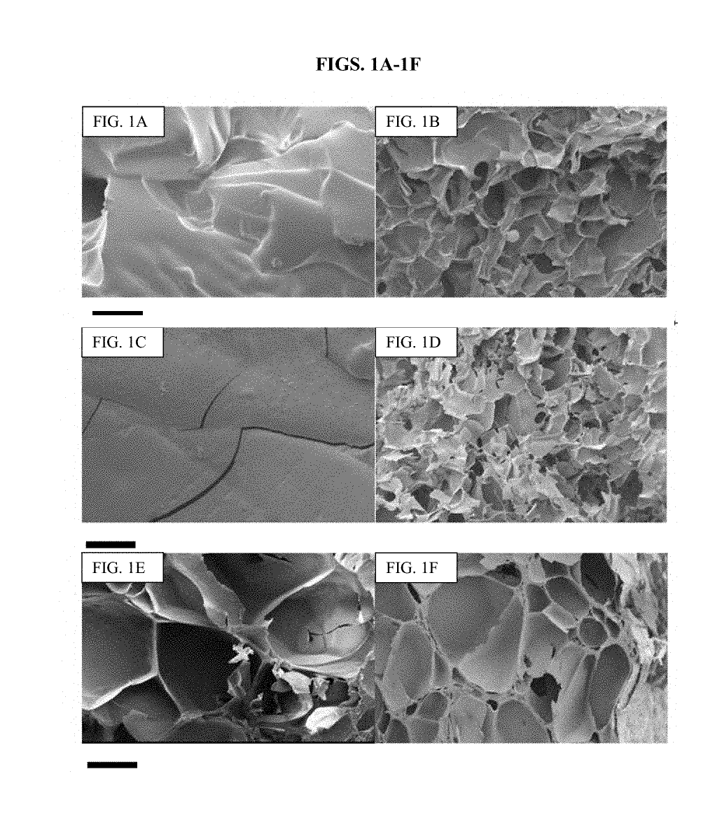

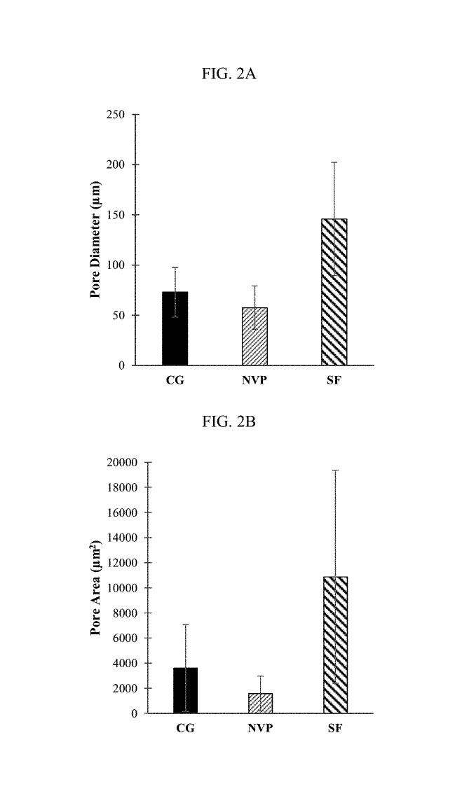

SEM images of the cryogel scaffolds and hydrogels are shown in FIGS. 1A-1F. ImageJ was used to measure both pore diameter and pore area of each scaffold type. The measurements of each scaffolds' average pore diameter and area are shown in FIGS. 2A & 2B.

As shown in FIGS. 2A & 2B, SF possessed both the largest pore diameter and area with average values of 146.03 μm and 10,873.07 μm², respectively. Previous literature has identified a pore diameter of at least 100 μm to be necessary for cellular infiltration and angiogenesis formation in bone applications. This diameter measurement falls above the required threshold of 100 μm for bone applications; however, it should be noted that while SEM analysis provide a solid representation of the surface of a scaffold, it provides little insight into the structure's interior. As such, ImageJ measurements taken on 2D representations of 3D structures carry little weight, and more advanced scaffold characterization techniques were also employed.

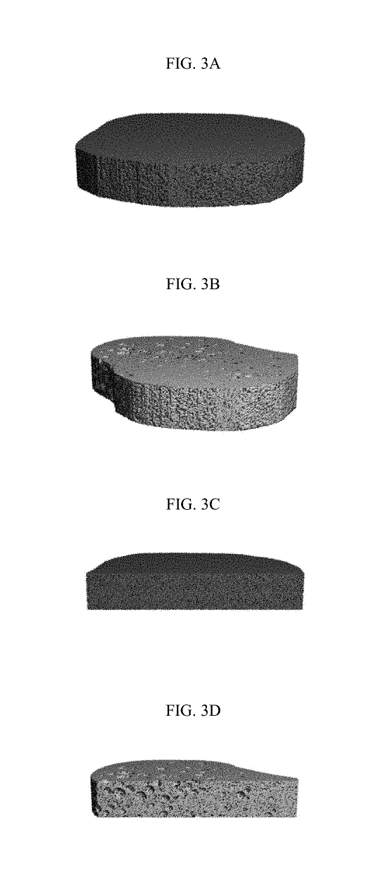

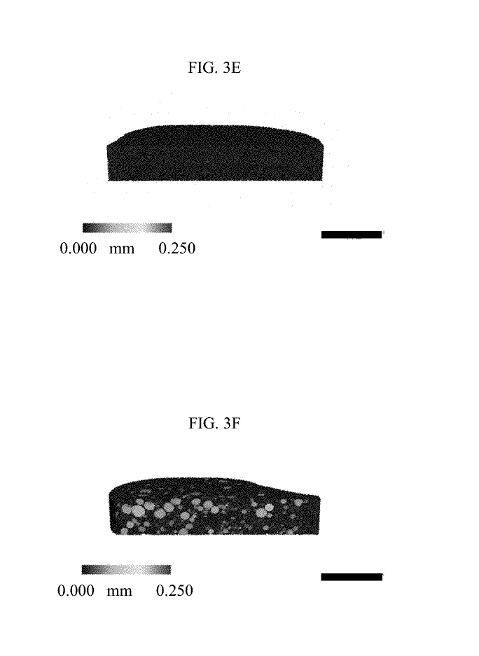

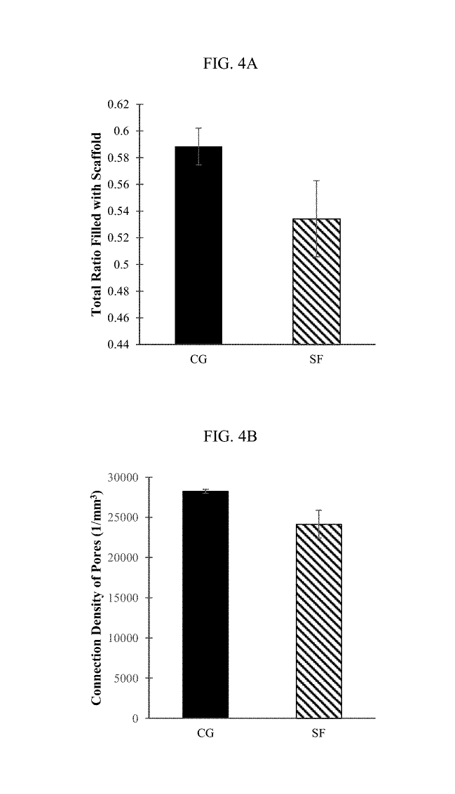

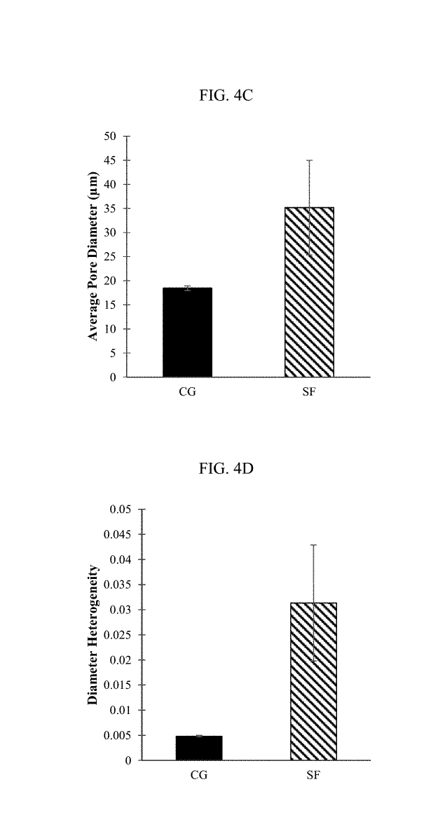

All types of cryogels were scanned with the μCT 35 described above. Only CG and SF cryogels had the stability to be scanned by the μCT, whereas the NVP cryogel fragmented when placed on the stand. Additionally, none of the hydrogel counterparts could be tested as their structures were not sturdy enough to fit on the stand and were composed of too much water. FIGS. 3Aa-3F show 3D reconstruction images of the CG and SF cryogels and their porosity. Note the variance in pore homogeneity between the CG and SF cryogels; where CG has a small, even distribution and SF has a much more variable distribution (FIGS. 3E & 3F). Overall, 58.5% of the total volume of the scaffold was filled with CG material, and 53.42% with SF material (FIG. 4A). The μCT reported much lower average pore diameters of 18.47 μm and 35.17 μm for CG and SF cryogels, respectively (FIG. 4C). The heterogeneity of these diameters was much larger for SF, with a standard deviation of 0.031 as opposed to 0.005 for CG. This shows a much larger variation in pore size throughout the scaffold, as also shown in all other methods of pore diameter measurement (FIG. 4D). Additionally, the average connection density of the pores was reported at 28,238.70 l/mm³ for CG and 24,146.50 l/mm³ for SF cryogels (FIG. 4B). This data suggests that while SF has the largest pore diameter, CG cryogels possess a slightly larger pore interconnectivity. This supports the ImageJ measurements with SF having the largest diameter, but a much smaller value was found with this measurement technique.

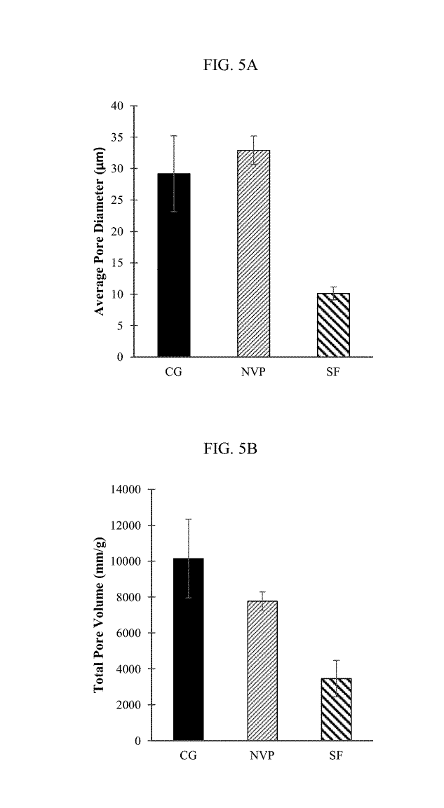

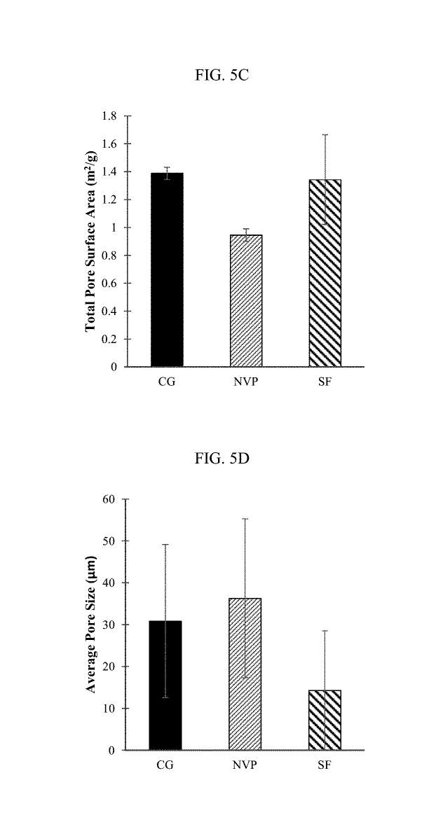

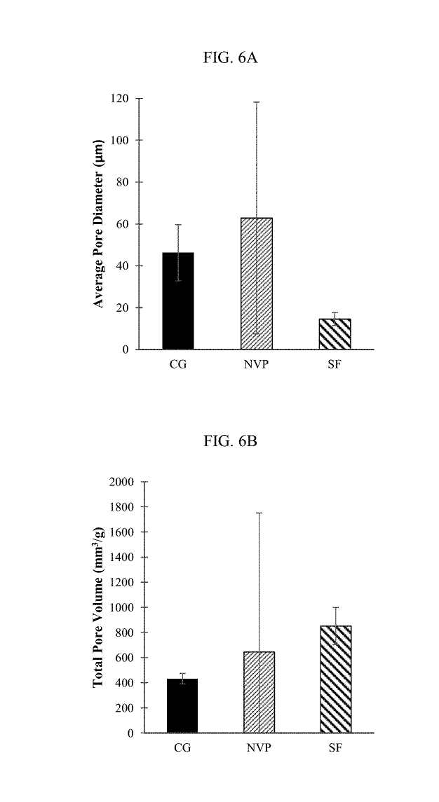

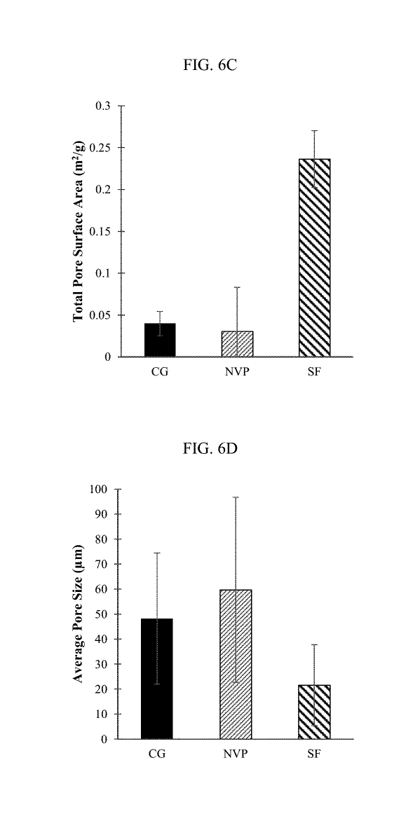

Mercury intrusion porosimetry was used as another method to analyze the various properties of the pores in the cryogels. Upon dehydration, NVP had the highest average pore diameter of 32.92 μm, followed by CG with 29.18 μm, and SF with 10.15 μm (FIG. 5A). The average pore diameter of the hydrated samples was highest for NVP with 62.83 μm, then CG with 46.27 μm, and lastly SF with 14.58 μm (FIG. 6A). All of these measurements being larger than the dry measurements. However, unlike the other pore measurement techniques, SF had the smallest and NVP the largest diameter compared with the other cryogels.

Next, the complete volume of the pores was examined for the dry samples with CG possessing the largest value of 10,144.50 mm³/g, NVP with 7,770.99 mm³/g, and SF with 3,459.42 mm³/g (FIG. 5B). For the hydrated samples, SF had the largest volume of 850.60 mm³/g, followed by NVP with 644.85 mm³/g, and CG with 423.87 mm³/g. All of these sample values are much smaller than the dry samples (FIG. 6B). Mercury intrusion porosimetry also provided the total pore surface area which, for the dry samples, was 1.39 m²/g for CG, 0.95 m²/g for NVP, and 1.34 m²/g for SF (FIG. 5C). For the hydrated samples, SF had the largest volume of 0.24 m²/g, followed by CG at 0.04 m²/g, and NVP at 0.03 m²/g, all of which are smaller than the dry samples (FIG. 6C). Lastly, the average pore size was also reported to be compared to previous methods of measurement. Here, there was an average pore size of 30.84 μm for CG, 36.28 μm for NVP, and 14.30 μm for SF (FIG. 5D). The hydrated samples pore size were 26.20 μm for CG, 37.04 μm for NVP, and 16.16 μm for SF (FIG. 6D).

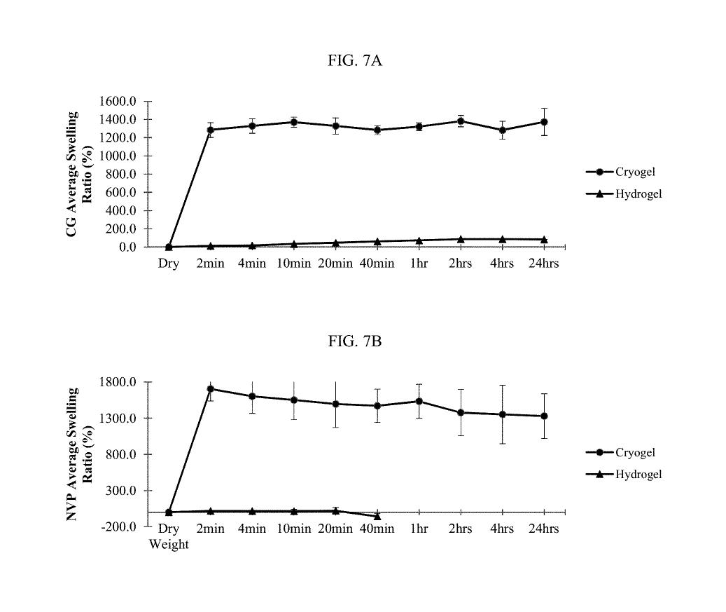

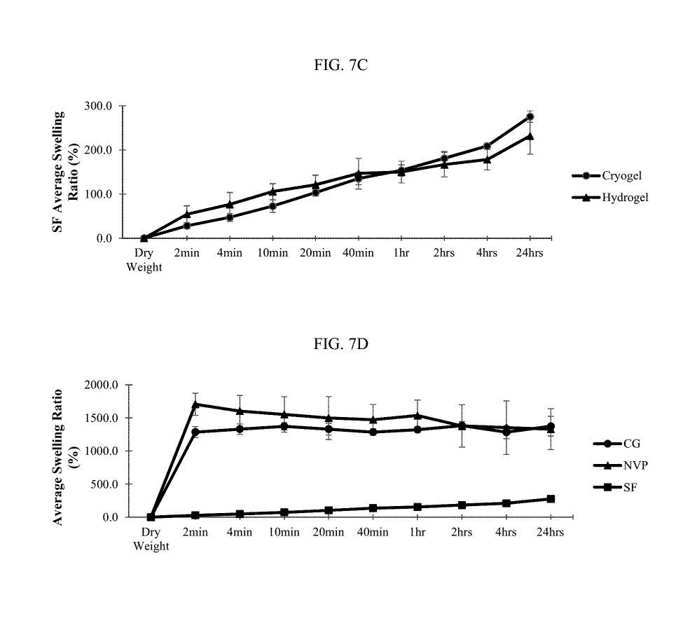

All cryogels and their hydrogel counterparts were completely dehydrated and then swollen over a period of time to examine their ability to return to their original morphology. All cryogels swelled to at least 275% of their original dry weight (FIGS. 7A-7C). The CG and NVP hydrogels demonstrated minimal amounts of swelling (FIGS. 7A and 7B); however, the SF hydrogel showed similar swelling ability to the SF cryogel (FIG. 7C). Note that by 40 minutes, the NVP hydrogels had broken down so drastically that a negative average swelling ratio (%) was recorded and after this time point, no further data could be collected (FIG. 7B). This shows a general superiority of cryogels to hydrogels for swelling upon rehydration to obtain their original morphology. When plotted against one another, CG and NVP cryogels reached their maximum swelling potential after two hours whereas SF cryogels reached their, significantly lower, swelling potential after 24 hours (FIG. 7D). The ability to swell is desirable for a tissue engineered bone graft for cellular infiltration through the absorption of its surroundings. This allows the cryogel to evenly distribute the cells throughout its structure as well as soak up any local growth factors and media. This also allows for the scaffold to swell and completely fill irregularly shaped defect sites.

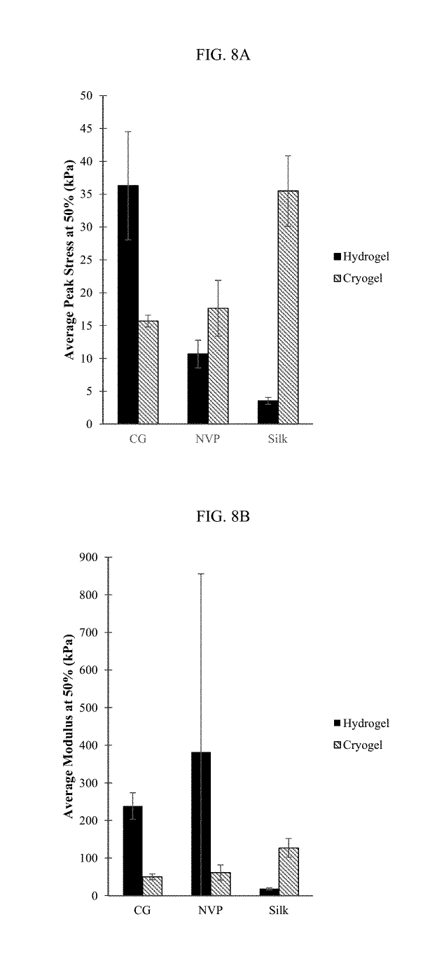

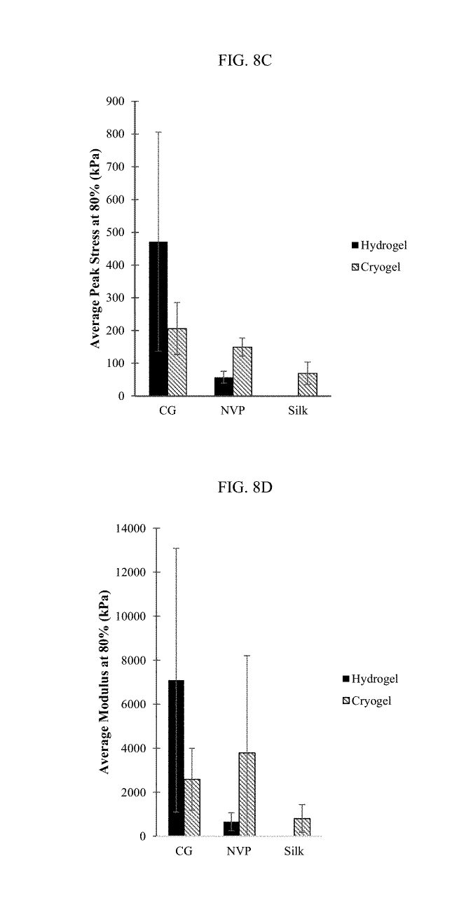

To further compare cryogels and hydrogels, ultimate compression testing was performed on cryogels and hydrogels of each material. At both the 50% and 80% strains, the SF cryogels and the CG hydrogels at 50% had the highest average peak stress showing their strength (FIGS. 8A and 8C). All hydrogels, other than SF, had a higher average modulus than the cryogels at 50% demonstrating the materials stiffness (FIG. 8B). At 80% ultimate compression, the NVP cryogel exhibited a higher modulus than its hydrogel counterpart, but CG hydrogels were still higher than CG cryogels (FIG. 8D). Additionally, SF hydrogels were not tested at 80% due to their complete loss of mechanical integrity at 50% compression. Since the hydrogels are largely composed of water, the structures were able to withstand high loads directly applied to the object, but then failed mechanically. By comparison, the spongy structure of the cryogels did not show as much resistance to compression, and allowed for the materials to return to their original shape when the load was removed. This property will be more evident through the cyclic loading data.

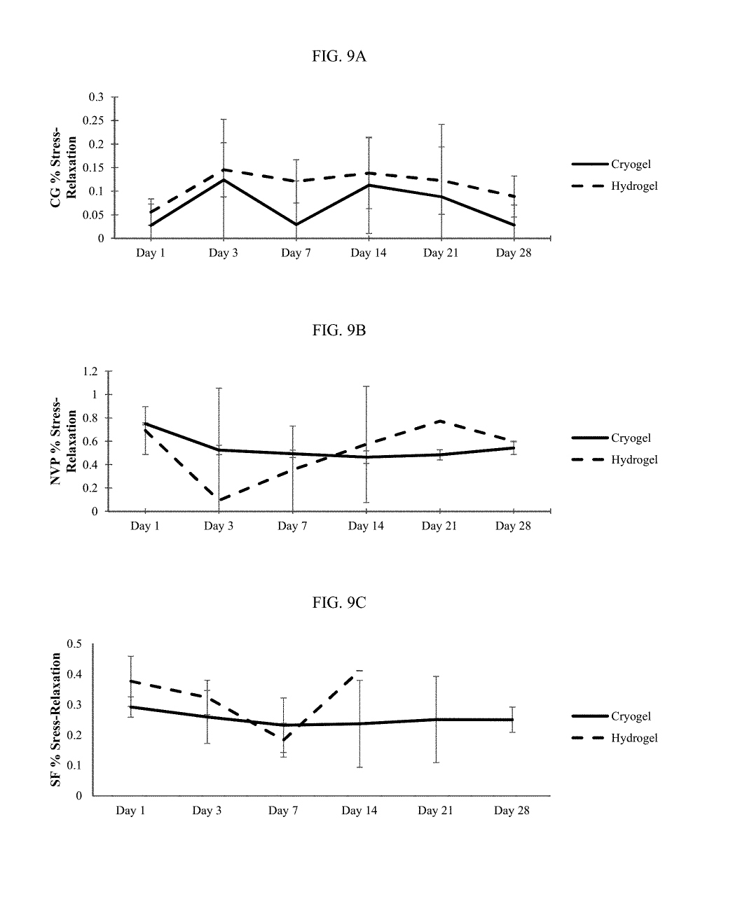

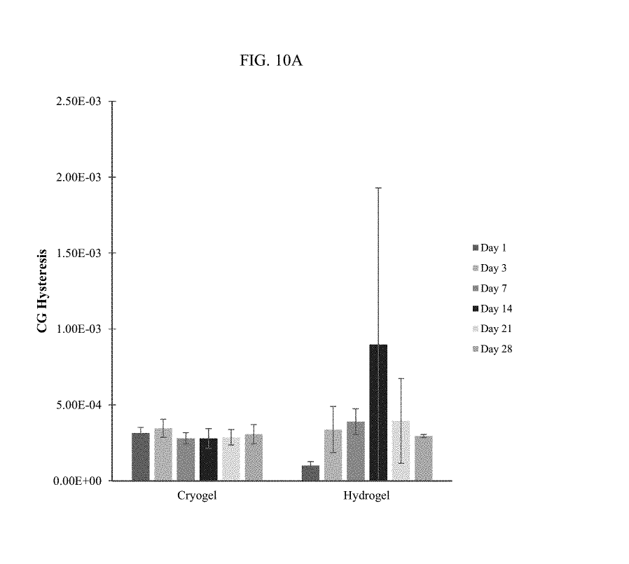

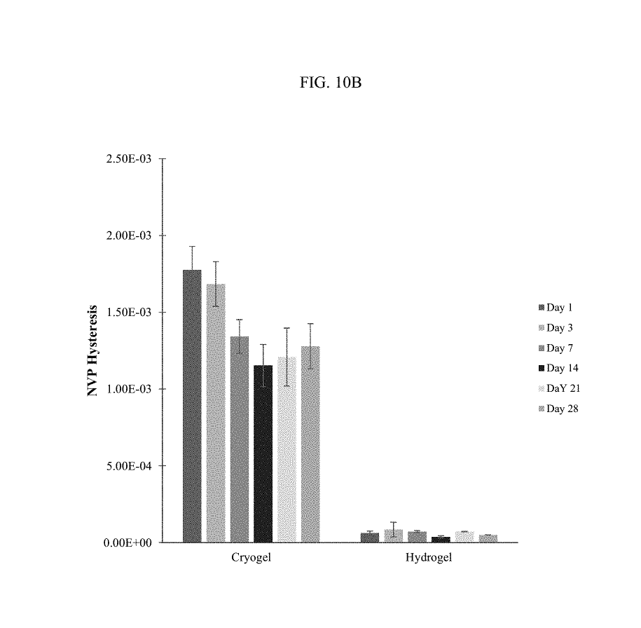

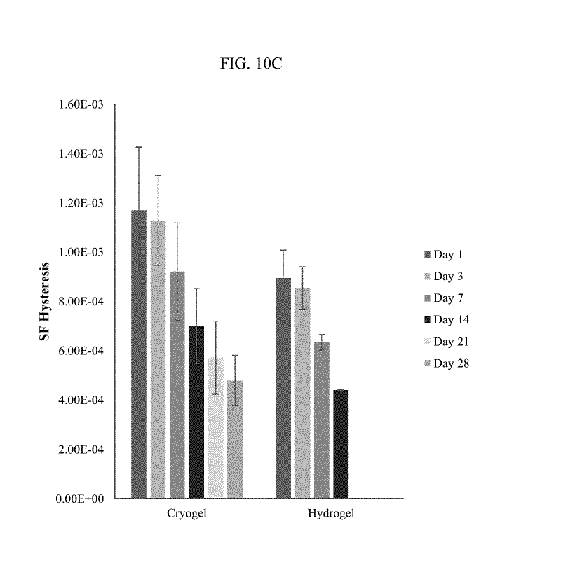

Cyclic loading on the MTS was used to compare the hydrogels and cryogels ability to withstand repeated application of a load and overall hysteresis. The percent stress-relaxation of each hydrogel and cryogel was recorded, providing further information on the overall change in structure. Higher values denote a larger deformation of the sample, demonstrating decreased resilience. All cryogels showed a generally lower percent stress-relaxation compared to their hydrogel counterparts (FIGS. 9A-9C). Note that all SF hydrogels were completely fragmented after day 14 (FIG. 9C). Additionally, the CG hydrogels reduced their thickness by approximately 25% thus becoming denser over the 28 days (FIG. 9A). This allowed them to withstand the cyclic loading better than expected and is not an accurate representation of CG hydrogel stress-relaxation and hysteresis. Hysteresis, or the loss of energy through loading and unloading, shows how well the structures were able to maintain their mechanical integrity over multiple load applications. The CG cryogels had a very low, constant hysteresis in comparison to the hydrogels (FIG. 10A). The NVP hydrogels showed a superior hysteresis to the cryogels (FIG. 10B) and the SF hydrogels and cryogels had very similar hysteresis (FIG. 10C). Overall, the SF hydrogels were completely fractured by day 14 and NVP hydrogels crumbled and did not hold their original shape. While NVP hydrogels showed superior hysteresis, they were not actually experiencing cyclic loading accurately. Additionally, all cryogels lasted the full 28 days and maintained their original shape and integrity, even with PBS degradation.

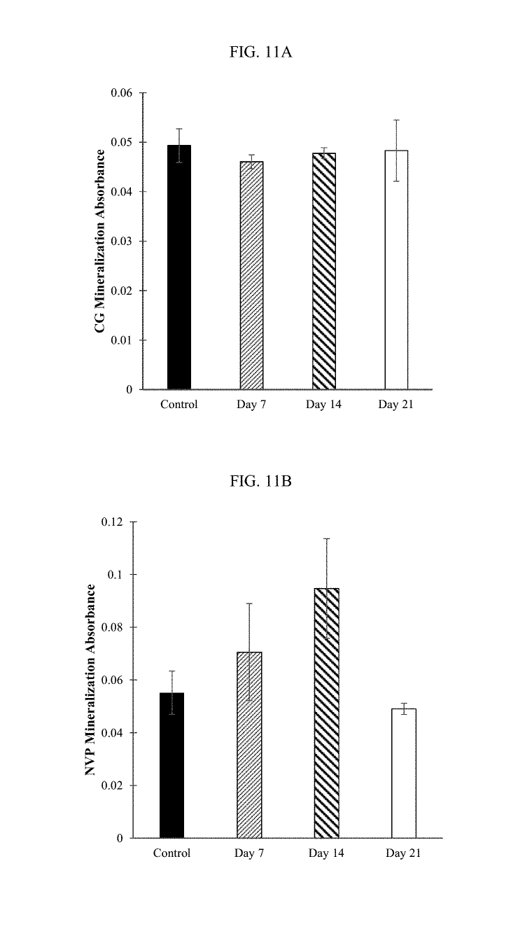

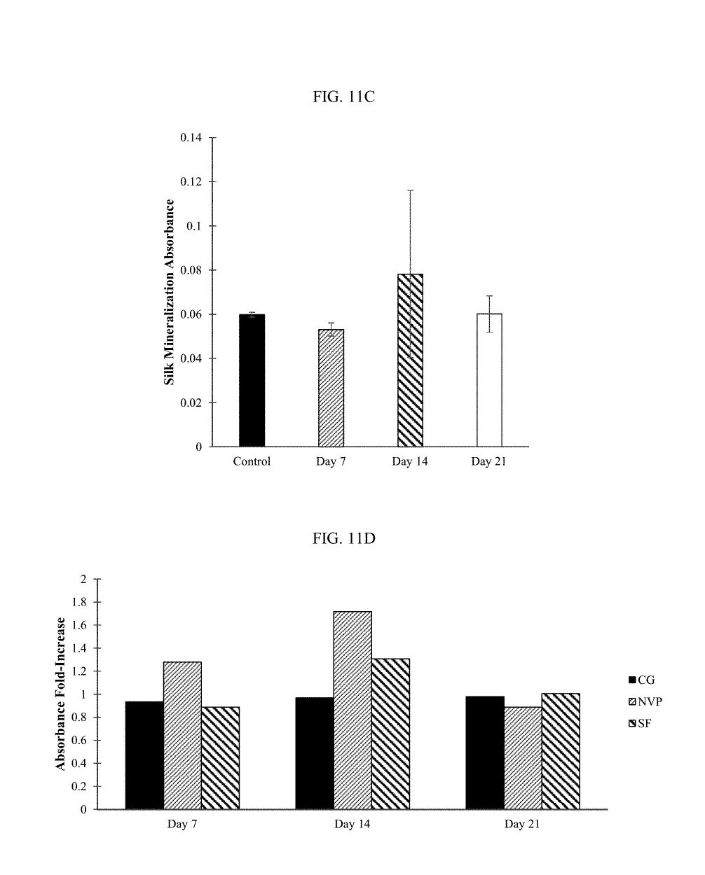

Upon mineralization for 7, 14, and 21 days, the acellular cryogel samples were stained with alizarin red stain (ARS) and absorbance was measured at 550 nm. The CG cryogels did not show any change in mineralization levels over 21 days (FIG. 11A). NVP and SF cryogels showed a slight increase in mineralization through day 14 and then a drop in absorbance levels (FIGS. 11B and 11C). The samples became so weak by day 21 that their fragmentation made it very difficult to accurately measure absorbance. The fold increase was calculated using the control as the initial value and plotted for all cryogels (FIG. 11D). When plotted on a single graph, it can be seen that all cryogels had essentially negligible mineralization over 21 days compared to the control materials (cryogels that were not exposed to simulated body fluid).

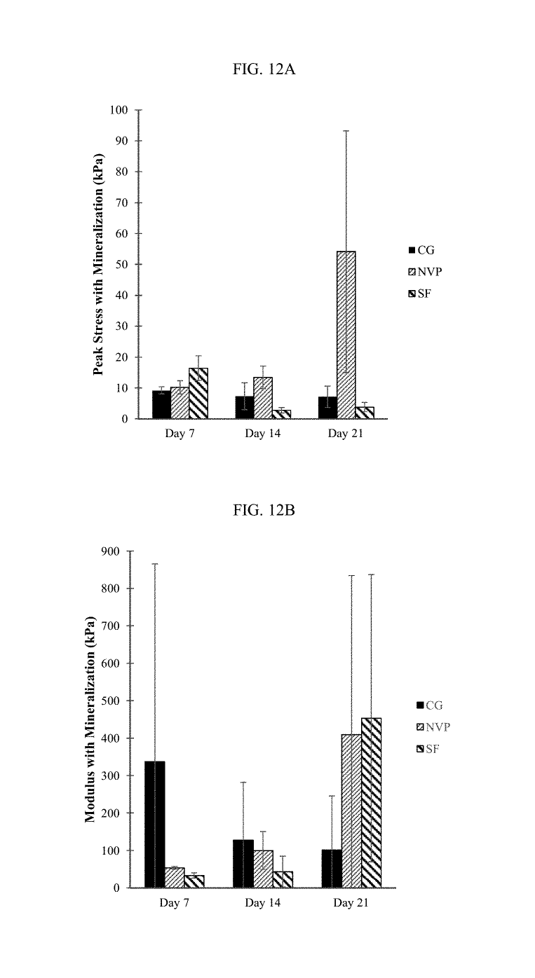

Ultimate compression at 50% was done on three of each type of cryogel (n=3) as shown in FIGS. 12A & 12B. CG cryogels had a fairly constant peak stress over all time points, supporting the previous data that these cryogels were not undergoing any sort of mineralization (FIG. 12A). NVP cryogels peak stress increased over the 21 days, while the SF cryogels decreased after only a week. The SF cryogels experienced some fragmentation which made it difficult to complete ultimate compression (FIG. 12A). Both NVP and SF cryogels increased their modulus over 21 days, suggesting a small amount of mineralization may have occurred and a corresponding increase in strength existed (FIG. 12B).

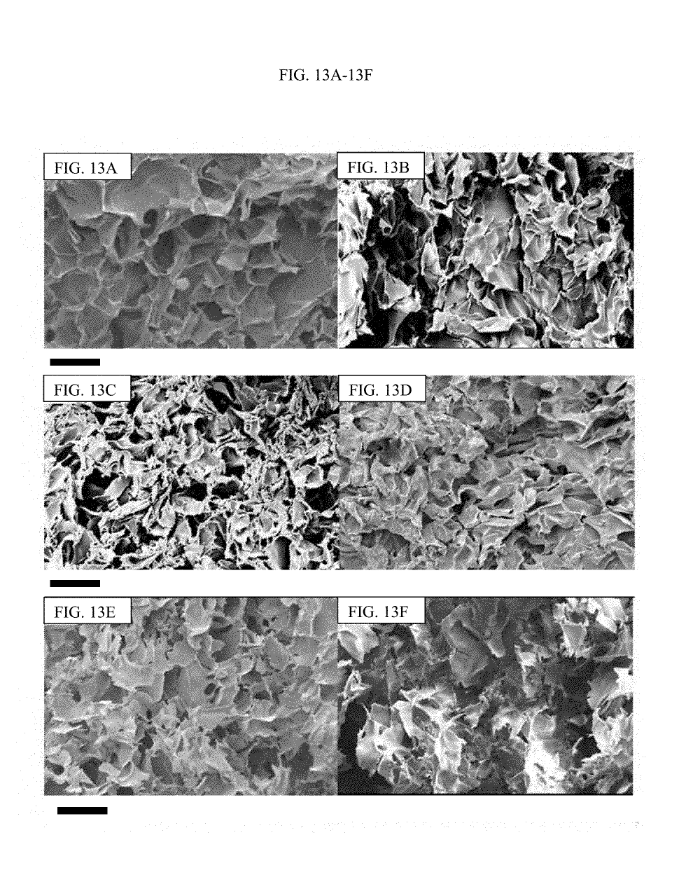

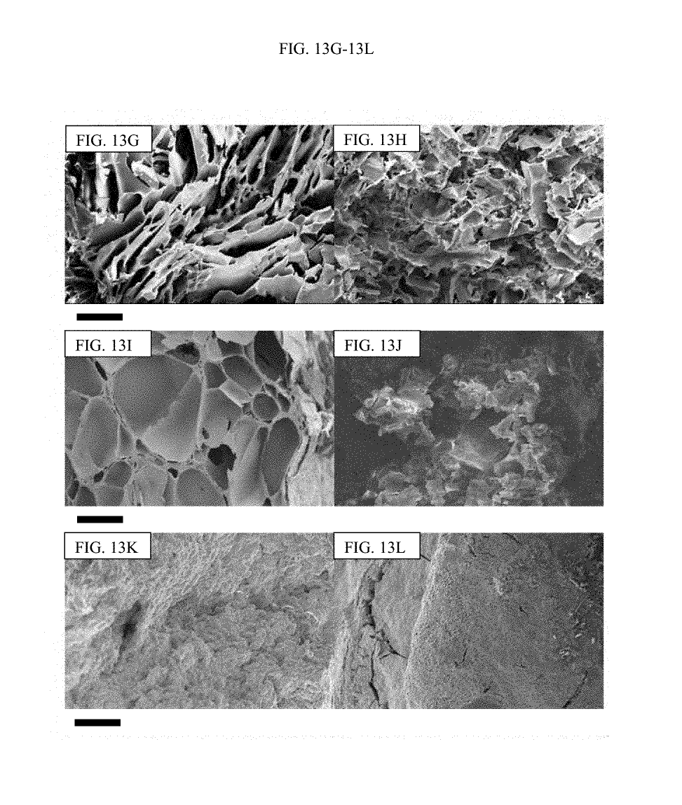

FIGS. 13A-13L shows SEM images of CG, NVP, and SF cryogels that were mineralized over 7, 14, and 21 days. By day 14, all cryogels showed a small amount of mineralization and once day 21 was reached, there was substantial mineralization on all material types.

### Example 2

In this Example, each of the cryogel scaffolds made in Example 1 were prepared with the addition of Manuka honey and analyzed for their structural and physical characteristics.

The chitosan/gelatin (CG) cryogel scaffolds were prepared as described in Example 1 with the addition of 0.5 mL of warm, sterile Manuka honey added to the solution including the chitosan with a syringe prior to adding the gelatin.

The N-vinyl-2-pyrrolidone (NVP) cryogel scaffolds were prepared as described in Example 1 with the addition of 0.5 mL of warm, sterile Manuka honey added with the Bis Acrylamide (Promega, Madison, Wis.).

The silk fibroin (SF) cryogel scaffolds were prepared as described in Example 1 with the addition of either 1% or 5% honey to the silk solution.

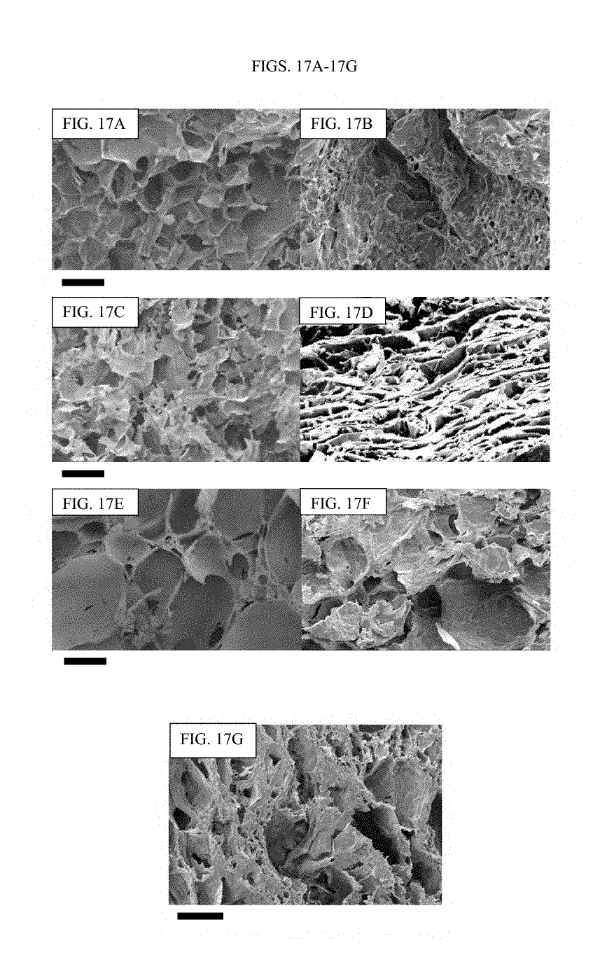

All cryogel scaffolds including honey are shown in FIGS. 17A-17G.

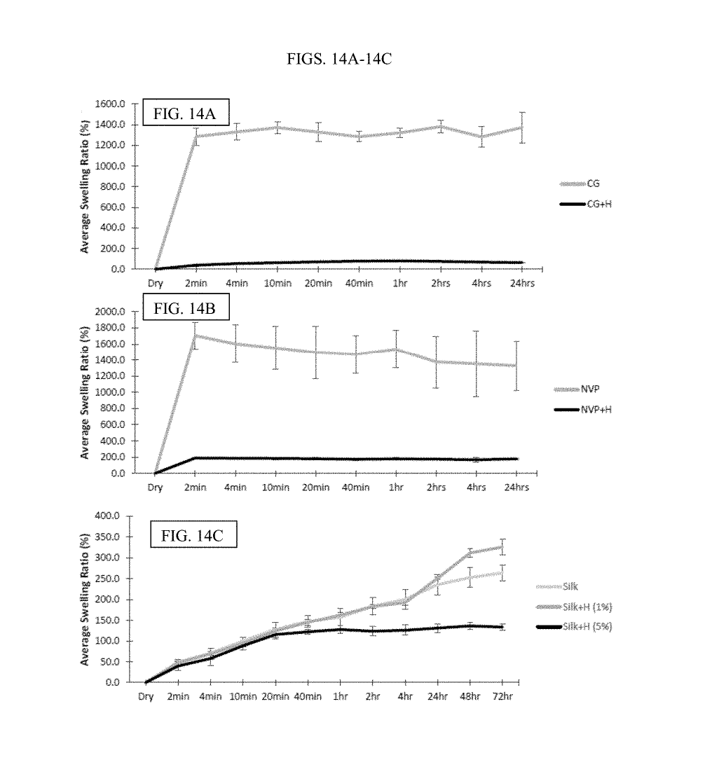

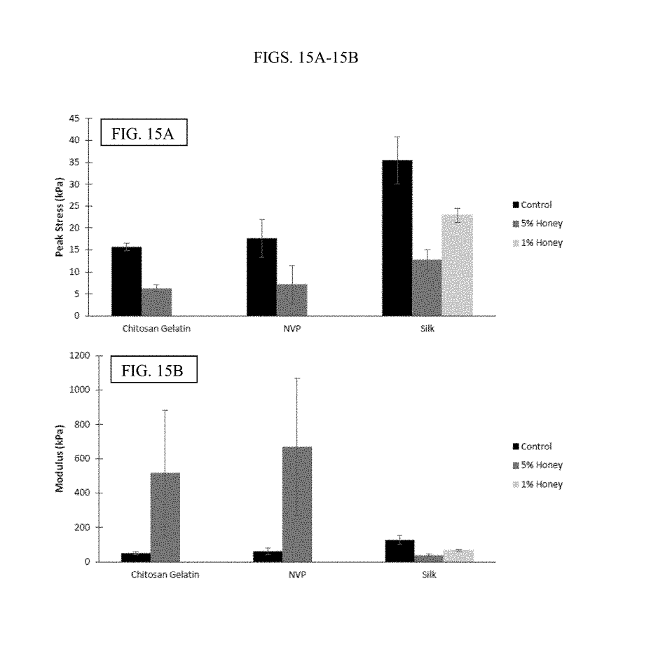

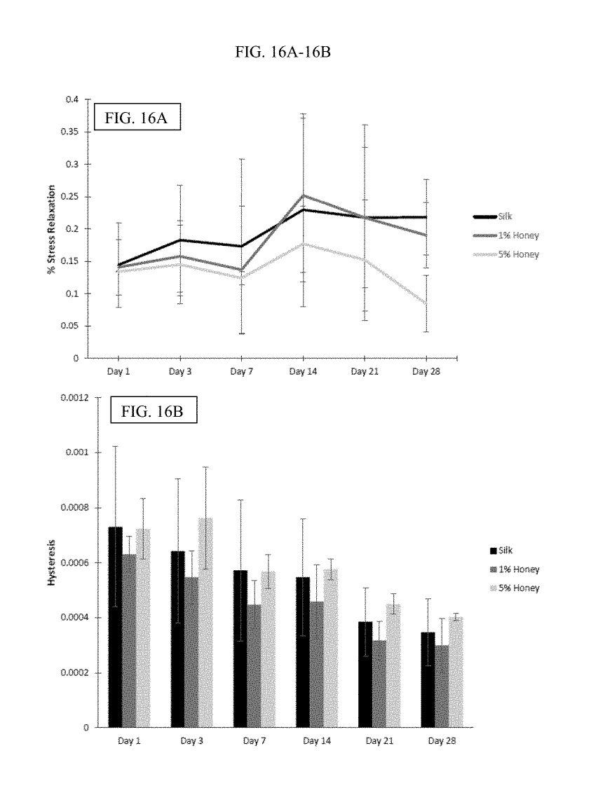

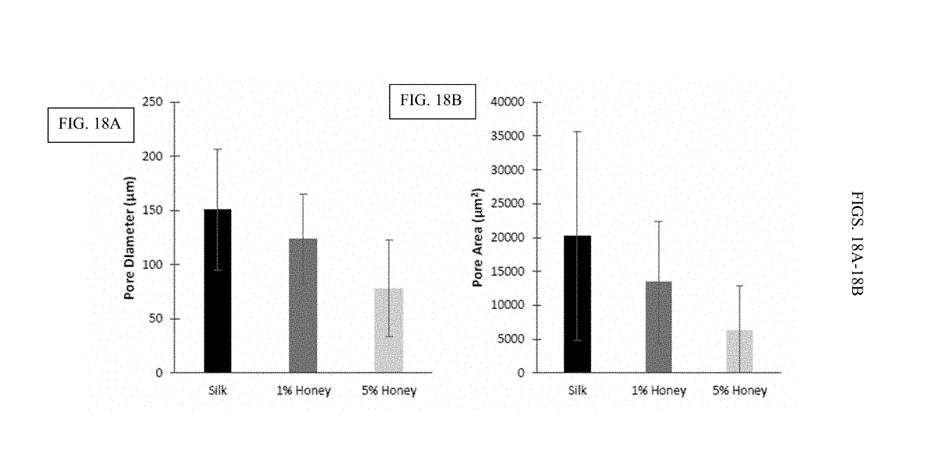

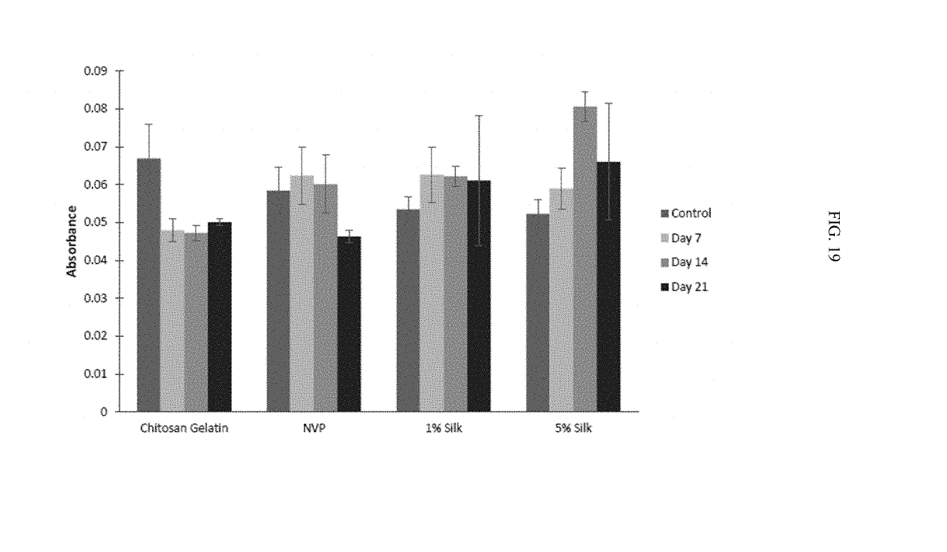

The following characteristics were analyzed as described above: average swelling ratio (FIGS. 14A-14C); ultimate compression (FIGS. 15A & 15B); stress-relaxation (FIG. 16A); hysteresis (FIG. 16B); pore diameter (FIGS. 18A & 18B); and mineralization (FIG. 19). Including honey in the various cryogels impacted the cryogels, which tended to be different based upon the different materials. Honey appeared to integrate well with the silk fibroin structures. With the silk fibroin structures, the inclusion of honey decreased the swelling potential, but did not adversely impact its mechanical properties.

### Example 3

In this Example, each of the cryogel scaffolds made in Example 1 were prepared with the addition of Manuka honey and mineralized, and analyzed for their structural and physical characteristics.

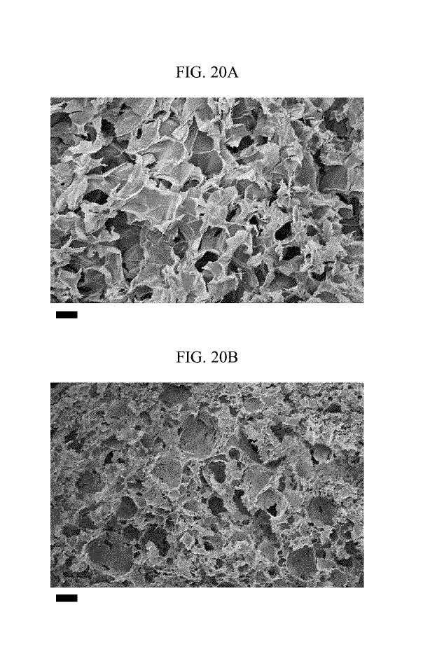

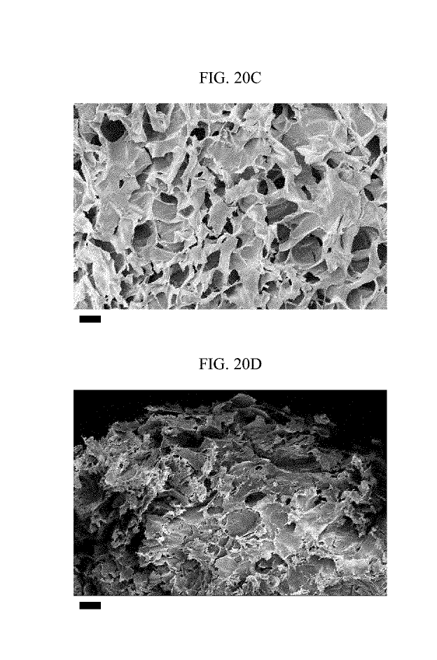

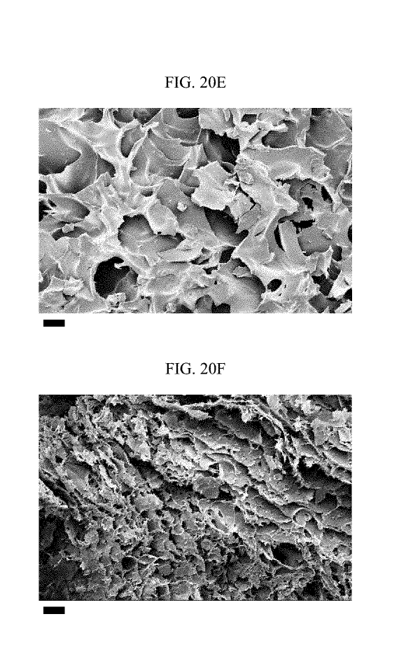

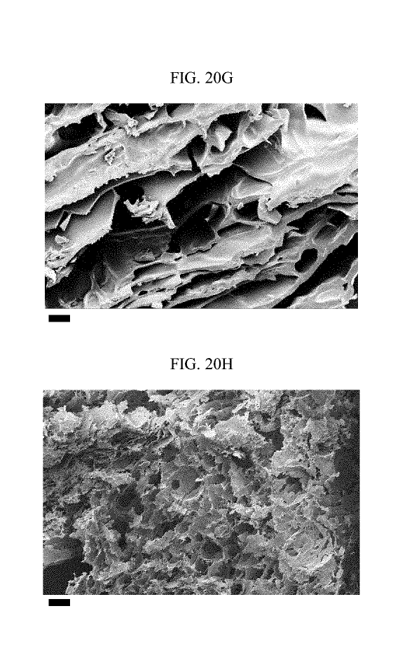

FIGS. 20A-20H depict scanning electron micrographs taken at 200× of a gelatin and SF cryogels, gelatin and silk fibroin (SF) cryogels with 1% honey (1H), gelatin and SF cryogels with 5% honey (5H), and gelatin and SF cryogel with 10% honey (10H).

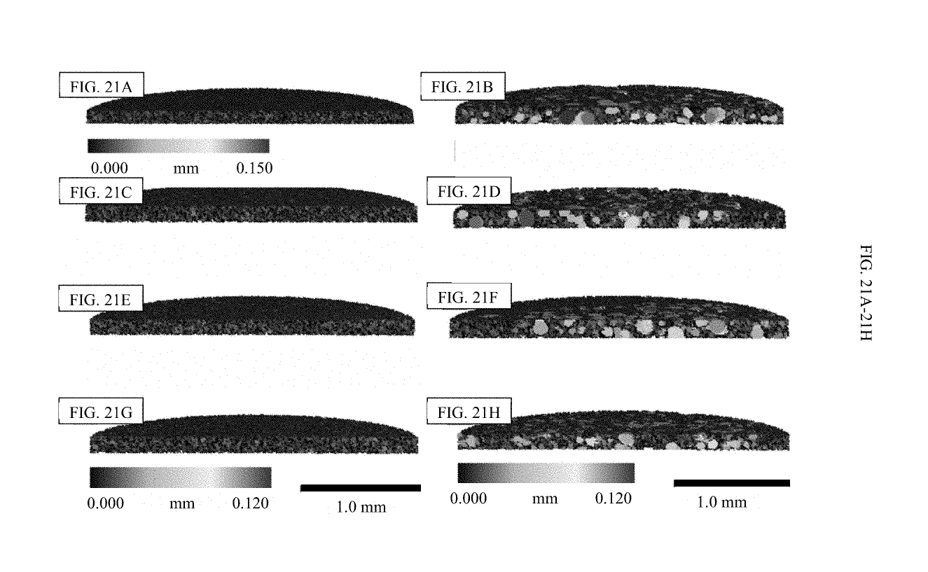

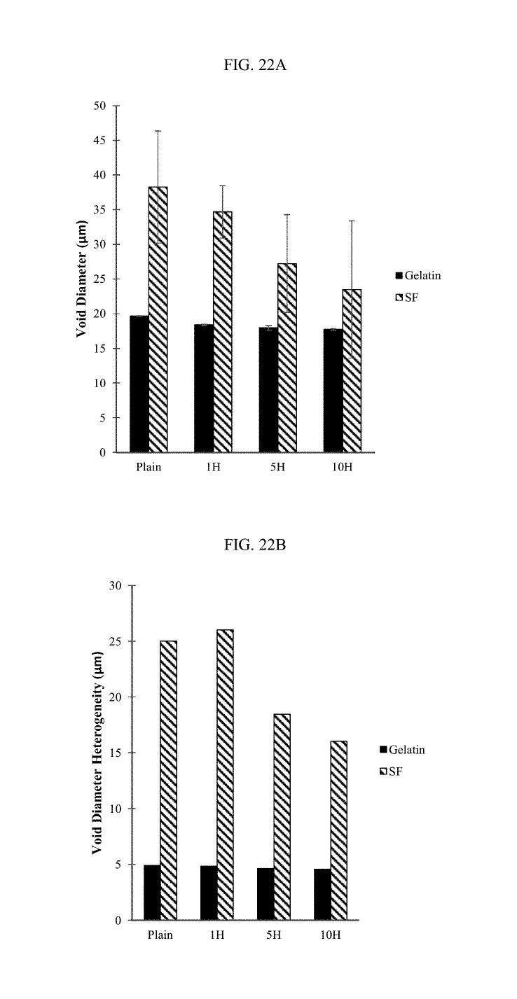

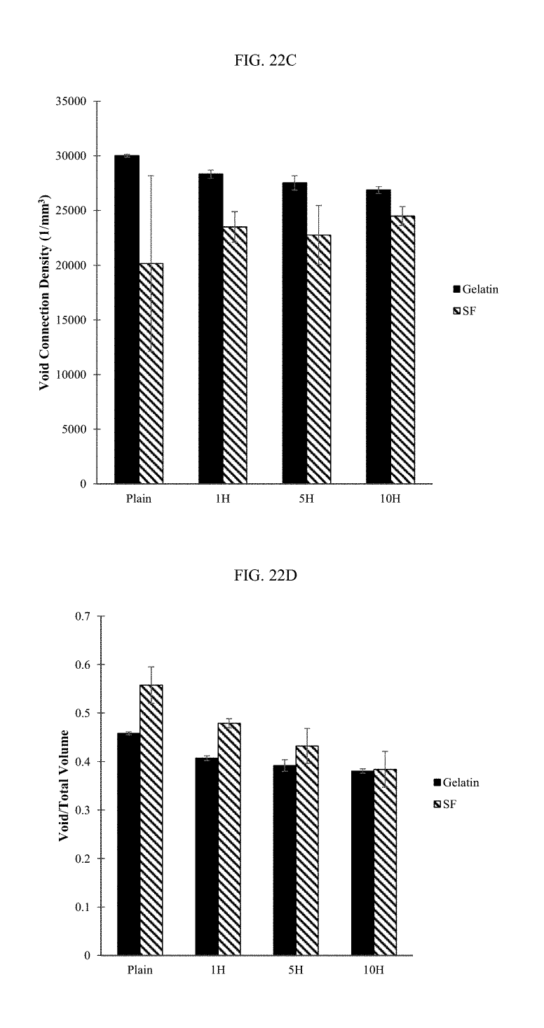

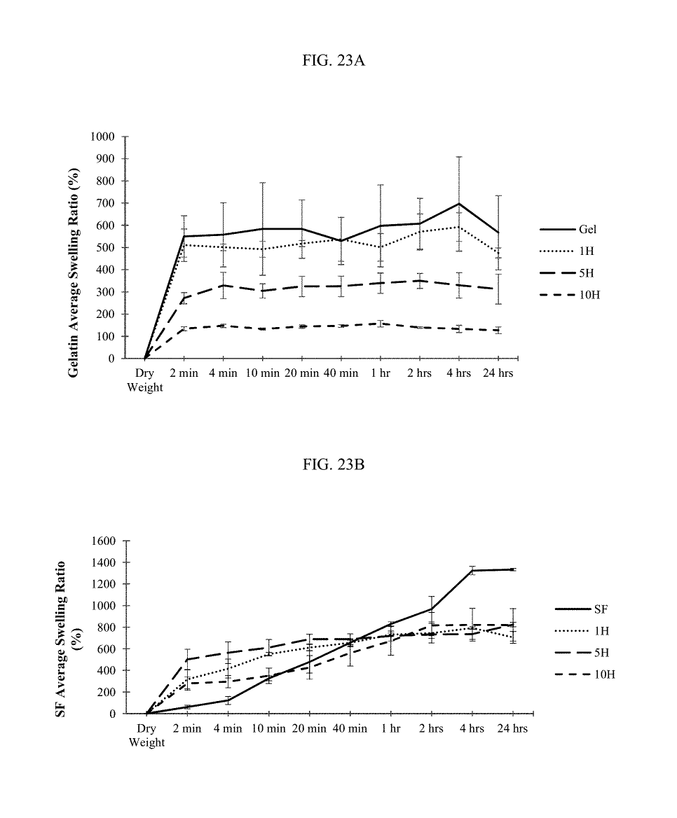

FIGS. 21A-21H depict μCT 3D reconstruction images of the cryogels where a sagittal cross section displays the inner pores for gelatin and SF cryogels, gelatin and SF cryogels with 1% honey (1H), gelatin and SF cryogels with 5% honey (5H), and gelatin and SF cryogel with 10% honey (10H). The shading bar denotes the pore size within each scaffold. Cryogels prepared with SF and SF with honey contained larger pore sizes than gelatin cryogels. As depicted in FIGS. 22A-22D, both SF and 1H SF had significantly larger pore diameters than all gelatin scaffolds. While SF cryogels showed a larger amount of variation in pore size, there is no significant difference between any of the scaffolds. Furthermore, gelatin cryogel connection density was significantly larger than SF. With respect to void/total volume, SF cryogels were significantly larger than all other scaffolds. Also, 1H SF was significantly larger than 1H, 5H, and 10H gelatin, as well as 10H SF. Lastly, gelatin was significantly larger than 5H gelatin, 10H gelatin, and 10H SF (p<0.05).

As depicted in FIGS. 23A and 23B, the average swelling ratio of gelatin cryogels with and without honey (FIG. 23A) was less than the average swelling ratio of SF cryogels with and without honey. As depicted in FIG. 23A, increasing honey in gelatin cryogels resulted in lower swelling. Swelling of SF cryogels with honey was also less than swelling of SF cryogels without honey (FIG. 23B).

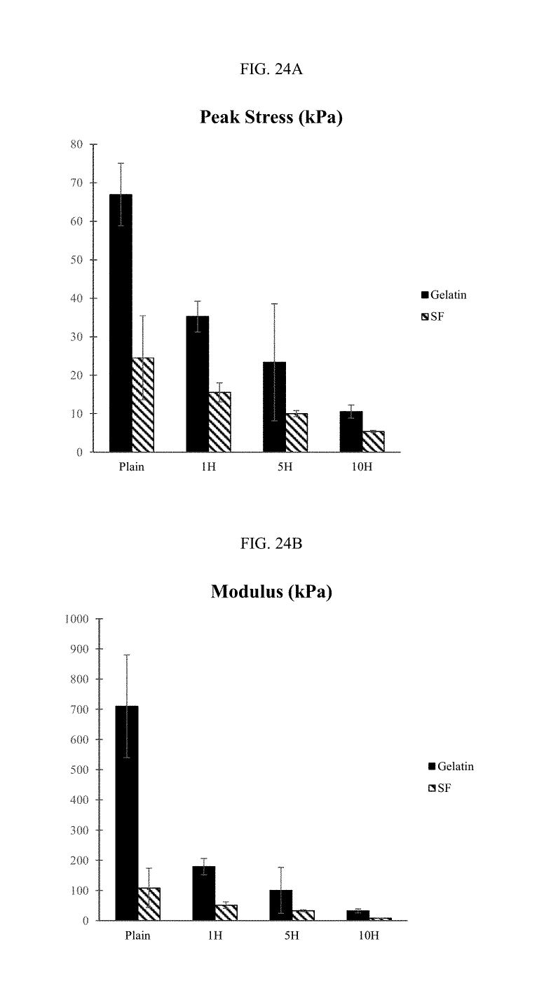

As depicted in FIGS. 24A and 24B, gelatin cryogels had a significantly larger average peak stress and modulus (kPa) than all other scaffolds. Additionally, 1H gelatin had a significantly larger average peak stress than 10H gelatin, as well as 5H and 10H SF.

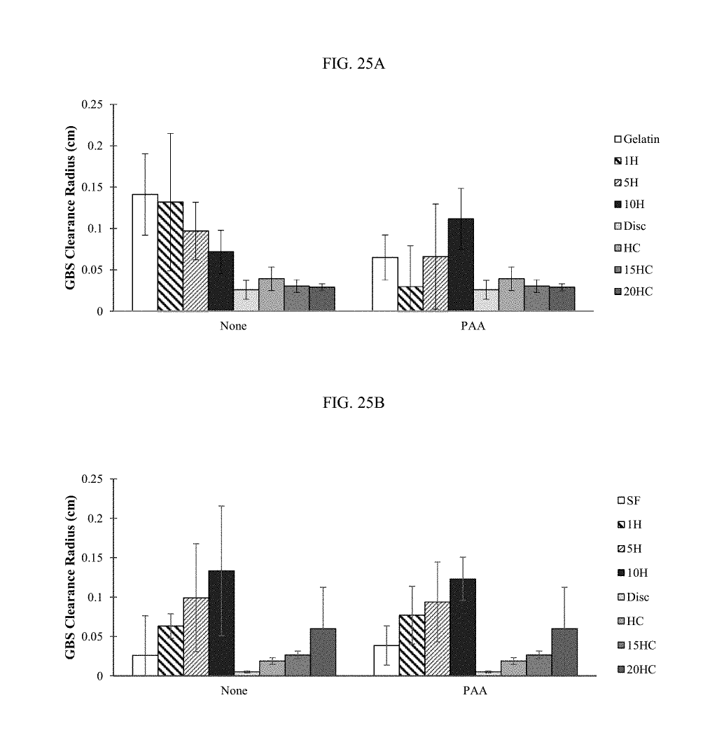

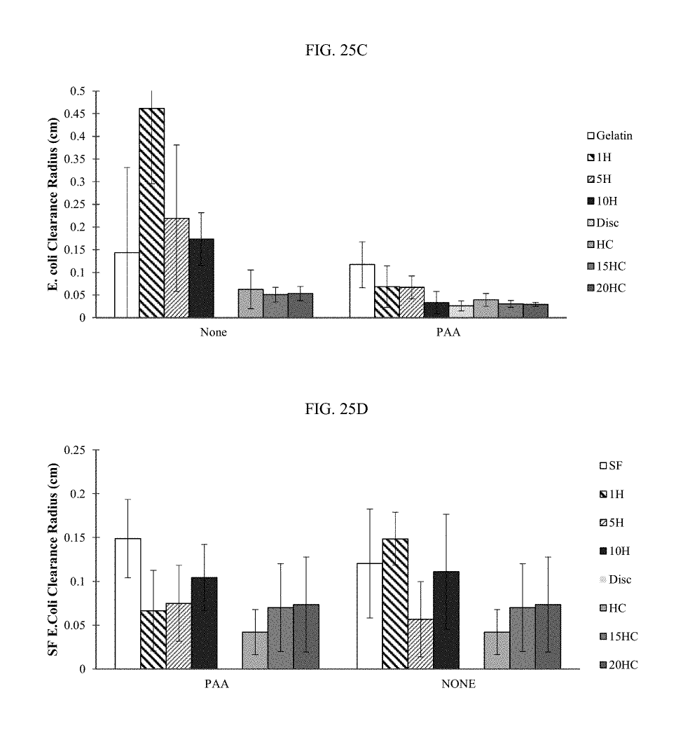

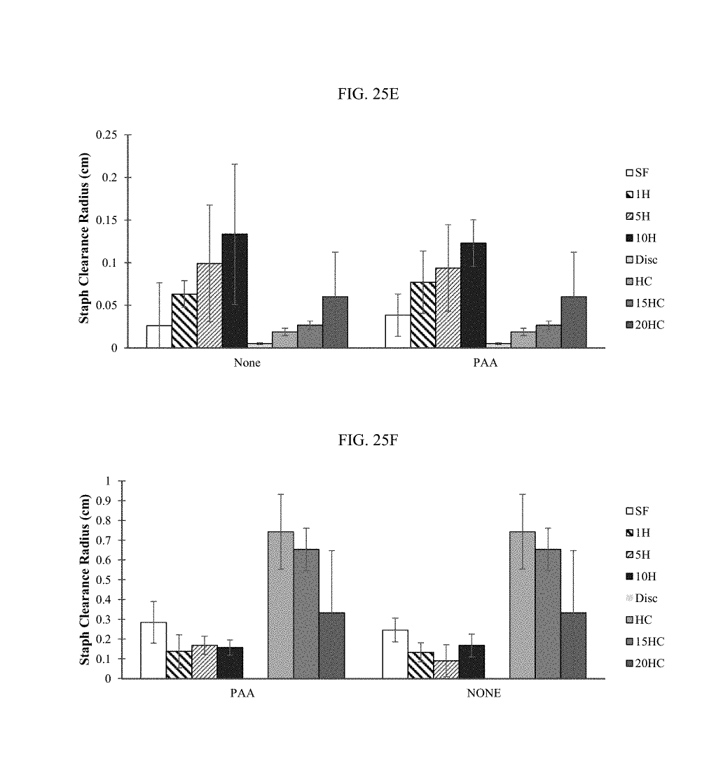

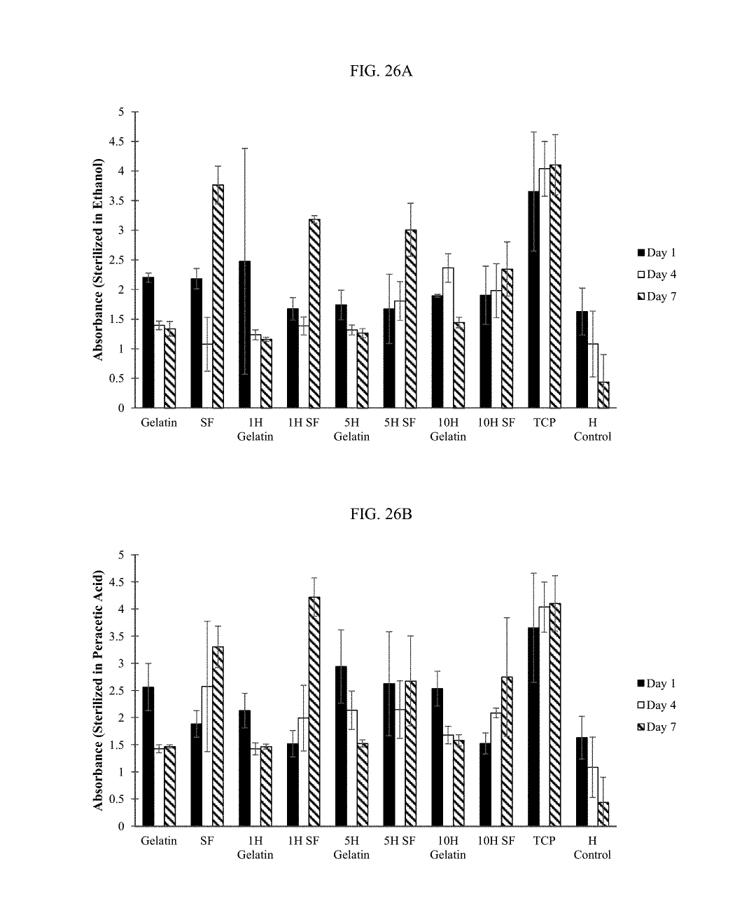

As depicted in FIGS. 25A-25F, clearance of Group B Streptococcus (GBS), Escherichia coli and Staphylococcus occurred in gelatin cryogels and silk fibroin (SF) cryogels having 1%-10% honey. Sterile discs with no honey (“Disc”) were used as controls. Additionally, Manuka honey controls (“HC”) of 1% to 20% honey were placed directly onto the bacteria.

As depicted in FIGS. 26A and 26B, of MG-63 cells proliferated on gelatin and SF cryogels with 1%-10% honey, and no honey where the scaffolds were sterilized in 70% ethanol (FIG. 26A) or peracetic acid (FIG. 26B). Note that TCP and honey media were used as controls.

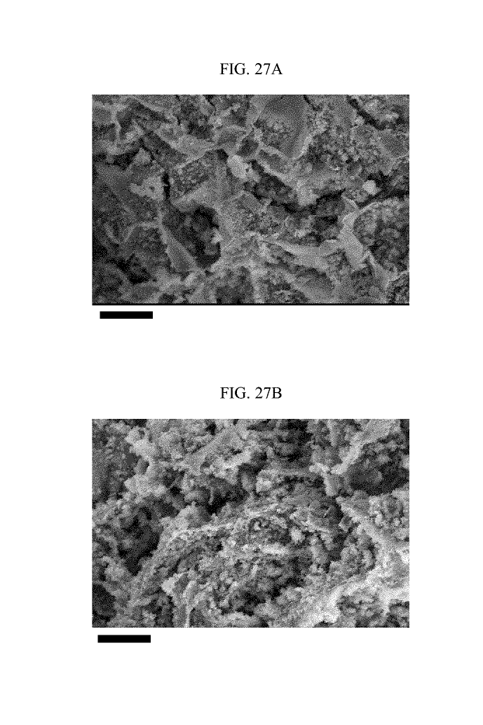

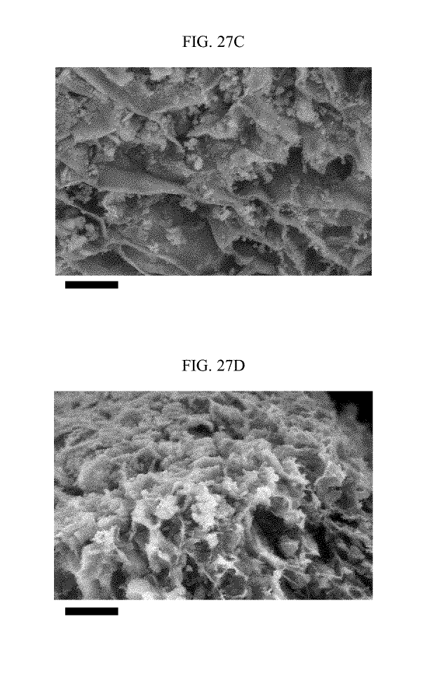

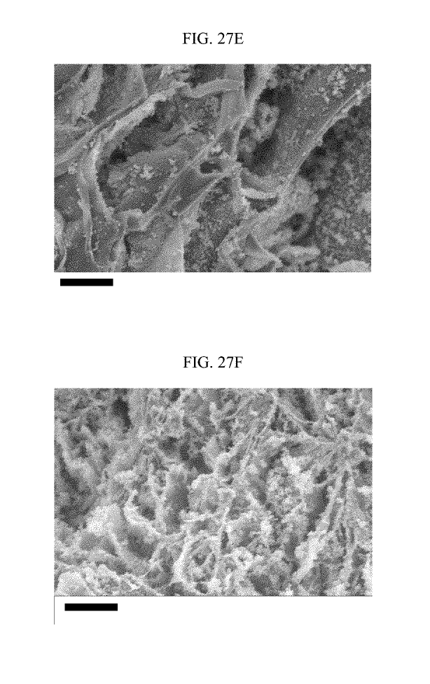

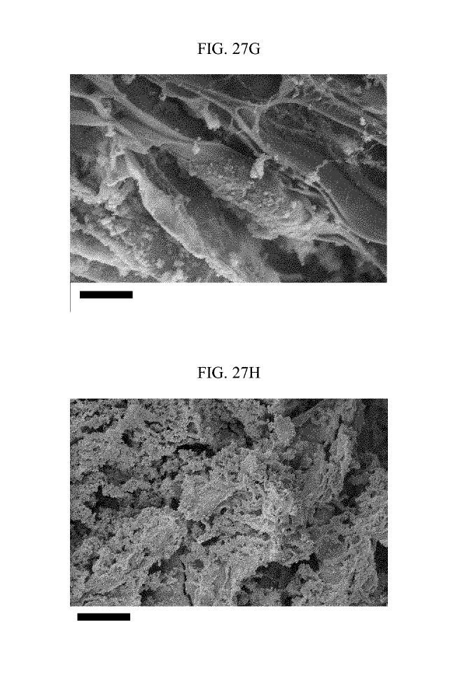

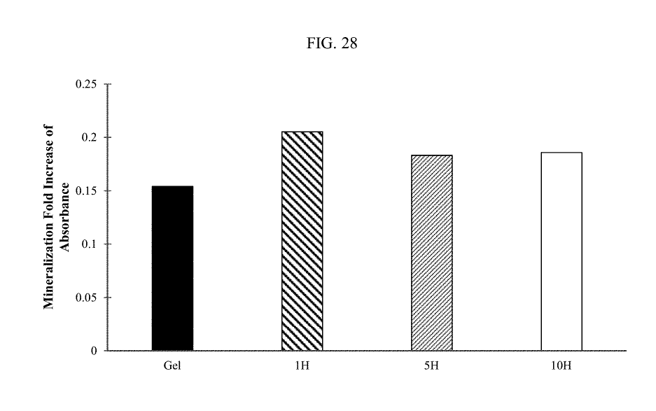

As depicted in FIGS. 27A-27H, SEM images taken at 500× showed mineralization of gelatin cryogels and SF cryogels having 1%-10% honey through dunking. As depicted in FIG. 28, incorporation of honey appeared to improve mineralization of gelatin cryogels. The amount of honey in gelatin cryogels also did not affect the amount of mineralization.

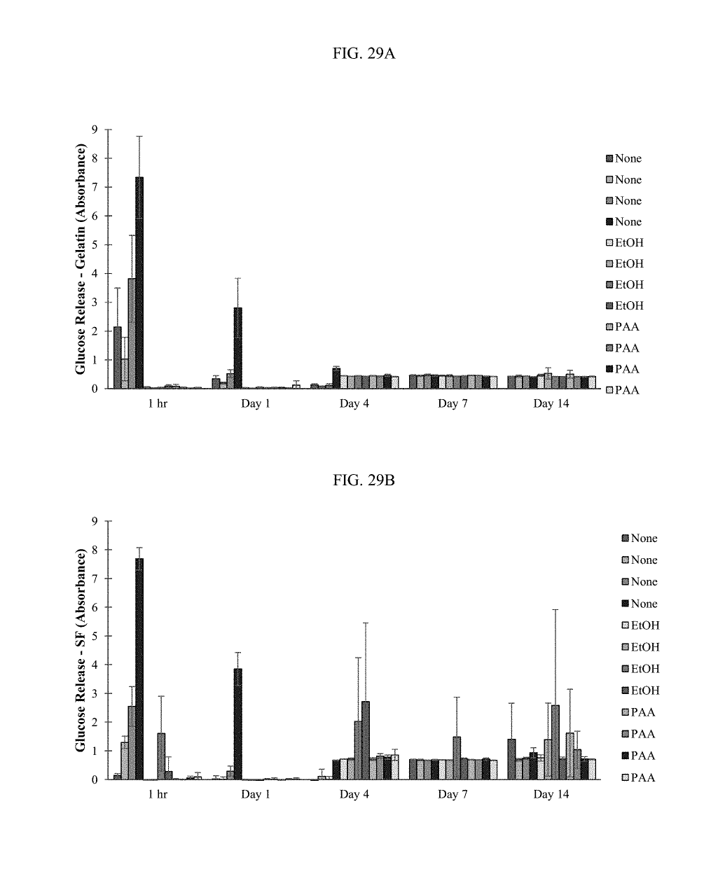

As depicted in FIGS. 29A and 29B, glucose was released from gelatin cryogels with honey (FIG. 29A) and SF cryogels with honey (FIG. 29B) over a 14 day time span.

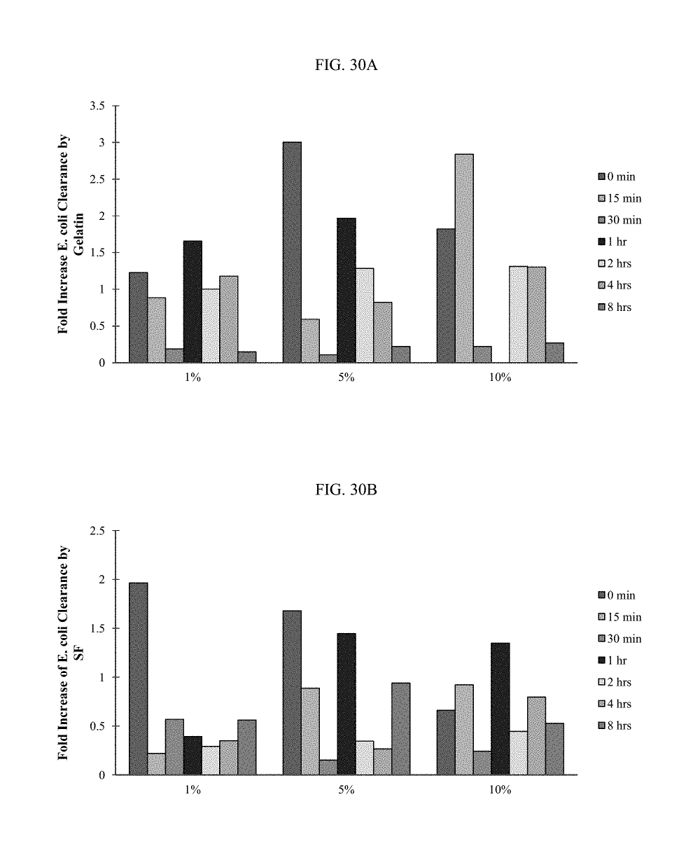

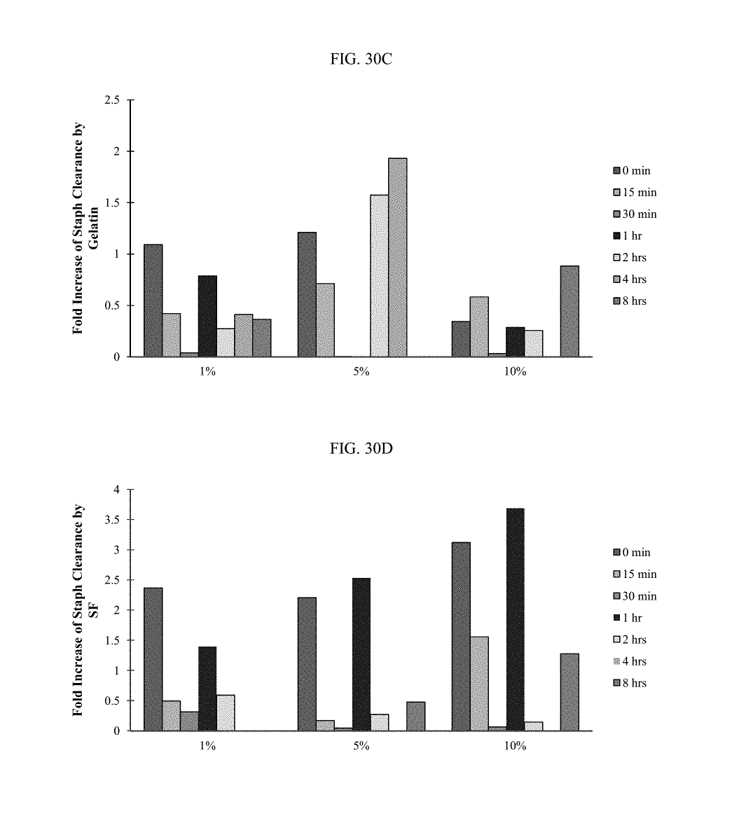

As depicted in FIGS. 30A-30D bacterial clearance in Escherichia coli (E. coli) and Staphylococcus (Staph) broth was effected by the presence of gelatin and SF cryogels incorporated with honey.

In view of the above, it will be seen that the several advantages of the disclosure are achieved and other advantageous results attained. As various changes could be made in the above devices and methods without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

1. A tissue engineered structure comprising a cryogel scaffold and honey.

2. The tissue engineered scaffold of claim 1 further comprising at least one biomolecule.

3. The tissue engineered scaffold of claim 2, wherein the at least one biomolecule is selected from the group consisting of a growth factor, a cytokine, a bioactive lipid, an immunoglobulin, and combinations thereof.

4. The tissue engineered scaffold of claim 3, wherein the at least one biomolecule is a preparation rich in growth factors.

5. The tissue engineered scaffold of claim 1, wherein the cryogel scaffold comprises a material selected from the group consisting of a synthetic polymer, a natural protein and combinations thereof.

6. The tissue engineered scaffold of claim 5, wherein the synthetic polymers is selected from the group consisting of polycaprolactone (PCL), polydioxanone (PDO), poly (glycolic acid) (PGA), poly(L-lactic acid) (PLA), poly(lactide-co-glycolide) (PLGA), poly(L-lactide) (PLLA), poly(D,L-lactide) (P(DLLA)), poly(ethylene glycol) (PEG), poly(ε-caprolactone) (PCL), montmorillonite (MMT), poly(L-lactide-co-ε-caprolactone) (P(LLA-CL)), poly(ε-caprolactone-co-ethyl ethylene phosphate) (P(CL-EEP)), poly[bis(p-methylphenoxy) phosphazene] (PNmPh), poly(3-hydroxybutyrate-co-3-hydroxyvalerate) (PHBV), poly(ester urethane) urea (PEUU), poly(p-dioxanone) (PPDO), polyurethane (PU), polyethylene terephthalate (PET), poly(ethylene-co-vinylacetate) (PEVA), poly(ethylene oxide) (PEO), poly(phosphazene), poly(ethylene-co-vinyl alcohol), a polymer nanoclay nanocomposite; a halogenated polymer solution containing metal compounds (e.g., graphite), poly(ethylenimine), grafted cellulosics, poly(ethyleneoxide), and poly vinylpyrrolidone, polystyrene (PS) and combinations thereof.

7. The tissue engineered scaffold of claim 5, wherein the natural protein is selected from the group consisting of silk fibroin, collagen, elastin, hyaluronic acid, gelatin, fibrinogen, chitin, chitosan, fibronectin and combinations thereof.

8. The tissue engineered scaffold of claim 1, wherein the honey is Manuka honey.

9. The tissue engineered scaffold of claim 1, further comprising a cell adhesion molecule.

10. The tissue engineered scaffold of claim 9, wherein the cell adhesion molecule is selected from the group consisting of fibronectin, vitronectin, collagen, an RGD (arginine-glycine-aspartic acid) peptide, a LDV (leucine-aspartic acid-valine) peptide, laminin and combinations thereof.

11. A method of preparing a tissue engineered structure comprising a cryogel scaffold and honey, the method comprising
preparing a solution comprising honey and cryogel scaffold material;
freezing the solution; and
thawing the frozen solution.

12. The method of claim 11, further comprising adding at least one biomolecule to the solution.

13. The method of claim 11, wherein the honey is Manuka honey.

14. The method of claim 11, wherein the cryogel scaffold material is selected from the group consisting of a synthetic polymer, a natural protein and combinations thereof.

15. The method of claim 11 further comprising re-freezing the thawed frozen solution and then thawing the re-frozen solution.

16. The tissue engineered structure of claim 1, further comprising a mineral.

17. The tissue engineered structure of claim 1, wherein the cryogel scaffold is selected from the group consisting of a chitosan and gelatin (CG) cryogel scaffold, a N-vinyl-2-pyrrolidone (NVP) cryogel scaffold, and a silk fibroin (SF) cryogel scaffold.

18. A method for promoting bone regeneration, the method comprising: implanting a tissue engineered structure comprising a cryogel scaffold.

19. The method of claim 18, wherein the cryogel scaffold is selected from the group consisting of a chitosan and gelatin (CG) cryogel scaffold, a N-vinyl-2-pyrrolidone (NVP) cryogel scaffold, and a silk fibroin (SF) cryogel scaffold.

20. The method of claim 18, wherein the cryogel scaffold comprises a mineralized cryogel scaffold.
